(19) United States
(12) Patent Application Publication
(10) Pub. No.: US 2011/0086338 A1
(43) Pub. Date: Apr. 14, 2011

(54) Bacteriophage/Quantum-Dot (Phage-QD) Nanocomplex to Detect Biological Targets in Clinical and Environmental Isolates

(76) Inventors: Jeeseong Hwang, Gaithersburg, MD (US); Rotem Edgar, Potomac, MD (US); Michael Mckinstry, Fairmont, WV (US); Gary Giulian, Gaithersburg, MD (US); Carl Merril, Bethesda, MD (US); Sankar Adhya, Gaithersburg, MD (US)

(21) Appl. No.: 11/884,604

(22) PCT Filed: Feb. 16, 2006
(86) PCT No.: PCT/US06/05537

§ 371 Date: Dec. 15, 2010

Related U.S. Application Data
(60) Provisional application No. 60/654,784, filed on Feb. 18, 2005.

(51) Int. Cl. C12Q 1/70; C12N 7/00

(52) U.S. Cl. 435/5; 435/235.1

(57) ABSTRACT

The invention is related to a non-biotinylated bacteriophage that comprises a nucleic acid sequence encoding a biotinylation domain, a complex that comprises a biotinylated bacteriophage and a biotin-specific ligand conjugated bioconjugate, and a method of detecting a bacterial cell in a sample comprising contacting the sample with a non-biotinylated bacteriophage that comprises a nucleic acid sequence encoding a biotinylation domain, wherein the bacteriophage is specific to the bacterial cell.

### RELATED APPLICATIONS

This application claims the benefit of U.S. Provisional Application No. 60/654,784 filed Feb. 18, 2005, which is hereby incorporated by reference in its entirety.

### FIELD OF THE INVENTION

The invention pertains to combining quantum dots (QDs) with engineered phage for the specific identification of biological targets including bacterial strain(s) or cells from clinical or environmental isolates.

### BACKGROUND OF THE INVENTION

The number and diversity of bacteriophages in the environment provide a promising natural pool of specific detection tools for pathogenic bacteria. Currently there are several methods for detection of pathogenic bacteria that exploit phage (McKinstry, M. & Edgar, R. in Phages: Their Role in Bacterial Pathogenesis and Biotechnology, eds. Matthew, K.-W., Friedman, D.-I., & Adhya, S.-L. (ASM Press, Washington, D.C. 2005), pp. 430-440): a plaque assay for detection of Mycobacterium tuberculosis (McNerney, R. et al. 2004 J Clin Microbiol 42:2115-2120); fluorescence labeled phage and immunomagnetic separation assay for detection of Escherichia coli (E. coli) O157:H7 (Goodridge, L. et al. 1999 Int J Food Microbiol 47:43-50; Goodridge, L. et al. 1999 Appl Environ Microbiol 65:1397-1404); phage-based electrochemical assays (Neufeld, T. et al. 2003 Anal Chem 75:580-585); a luciferase reporter mycobacteriophage and Listeria phage assays (Banaiee, N. et al. 2001 J Clin Microbiol 39:3883-3888; Loessner, M.-J. et al 1996 Appl Environ Microbiol 62:1133-1140); and detection of the phage-mediated bacterial lysis and release of host enzymes (e.g., adenylate kinase) (Blasco, R. et al. 1998 J Appl Microbiol 84:661-666).

Two limiting features when detecting pathogenic bacteria are sensitivity and rapidity. Common fluorophores (e.g., green fluorescence protein and luciferase) used as reporters have two major disadvantages: low signal-to-noise ratio due to auto-fluorescence of clinical samples and of bacterial cells and low photo-stability such as fast photobleaching. To overcome these disadvantages, we employed new fluorescent semiconductor nanocrystals, quantum dots (QDs) (Sukhanova A. et al. 2004 Anal Biochem 324:60-67). QDs are colloidal semiconductor (e.g., CdSe) crystals of a few nanometers in diameter. They exhibit broadband absorption spectra and their emissions are of narrow bandwidth with size-dependent local maxima. The presence of an outer shell of a few atomic layers (e.g., ZnS) increases the quantum yield and further enhances the photostability resulting in photostable fluorescent probes superior to conventional organic dyes. Recently, development in surface chemistry protocols allows conjugation of biomolecules onto these QDs to target specific biological molecules and probe nano-environments (Dubertret, B. et al. 2002 Science 298:1759-1762; Yao, J. et al. 2005 Proc Nati Acad Sci USA 102:14284-14289; Hahn, M.-A. et al. 2005 Anal Chem 77:4861-4869). The power to observe and trace single QDs or a group of bio-conjugated QDs, enabling more precise quantitative biology, has been claimed to be one of the most exciting new capabilities offered to biologists today (Michalet, X. et al. 2005 Science 307:538-544; Tokumasu, F. et al. 2005 J Cell Sci 118:1091-1098).

### SEGUE TO THE INVENTION

Typically, the detection of small numbers of bacteria in environmental or clinical samples requires an amplification step involving the growth of bacteria in culture to increase cell number. This procedure considerably prolongs the detection time, especially for slow growing bacteria. Here we report a sensitive, rapid and simple method for detection of bacteria. This method combines in vivo biotinylation of engineered host-specific bacteriophage and conjugation of the phage to streptavidin-coated quantum dots. This phage-based assay reduces the “amplification” to a short time (5 to 20 min from infection to lysis) since each infected bacterium can result in a release of 10-1000 phage that can be readily detected by the use of QDs.

### SUMMARY OF THE INVENTION

The invention is related to a non-biotinylated bacteriophage that comprises a nucleic acid sequence encoding a biotinylation domain.

The invention is also related to a complex that comprises a biotinylated bacteriophage, and a biotin-specific ligand conjugated bioconjugate.

The invention is further related to a method of detecting a bacterial cell in a sample comprising contacting the sample with a non-biotinylated bacteriophage that comprises a nucleic acid sequence encoding a biotinylation domain, wherein the bacteriophage is specific to the bacterial cell.

### BRIEF DESCRIPTION OF THE DRAWINGS

FIG. 1. An overall strategy of bacterial detection using nano-engineered phage-QD complexes. (a) A schematic representation of the detection (see text for details). (b) Western blot analysis of T7-bio or control T7-myc phage particles using streptavidin-HRP.

FIG. 2. T7-bio phage bound to streptavidin functionalized QDs. TEM images of phage or phage-QD targeted bacteria. The symbol ∇ points to QD conjugated to the phage head. The inset shows control T7-myc phage that are not biotinylated and therefore have no conjugated QDs. Scale bars are 50 nm.

FIG. 3. Phage-QDs complexes detected by Flow cytometry. Scatter plots of bacteria targeted with T7-myc (a) or T7-bio (b) phage after addition of QDs. (c) Histograms of the number of cells vs. fluorescence with control phage (T7-myc) and biotinylated phage (T7-bio) and comparison of percentages within P2 range and medians of the fluorescence intensities calculated from the histograms.

FIG. 4. Fluorescence microscope images of phage-QD complexes bound to cells. (a, b) A typical picture of E. coli cells exposed at low multiplicity to QD-tagged biotinylated phage. The field was simultaneously illuminated with a low-intensity white light source and a fluorescence excitation (447±15 nm). The images are of two different quantized blinking states of a single QD: (a) “off” and (b) “on”. (c) Fluorescence micrograph of cells with 100-fold excess of biotinylated phage. (d) Bright field transmission micrograph of the same sample area, obtained immediately after capturing the image in (c). Note that some cells are immobilized on a substrate, but some are mobile in solution resulting in out-of-focus fluorescence images when the focus is maintained on the cells on a substrate surface. Scale bars are 1 μm (a, b) or 2 μm (c, d).

FIG. 5. Bio-conjugated Semiconductor Nanocrystals. Merits of Semiconductor Nanocrystals: Size-Dependent Luminescence, Broad Excitation, Narrow and Symmetrical Emission, Brightness, Stable Photoluminescence, High Extinction Coefficient, Biocompatibility, Chemical Sensitivity. A family of QD particles can be made to emit a full spectrum of colors when excited with a single excitation source.

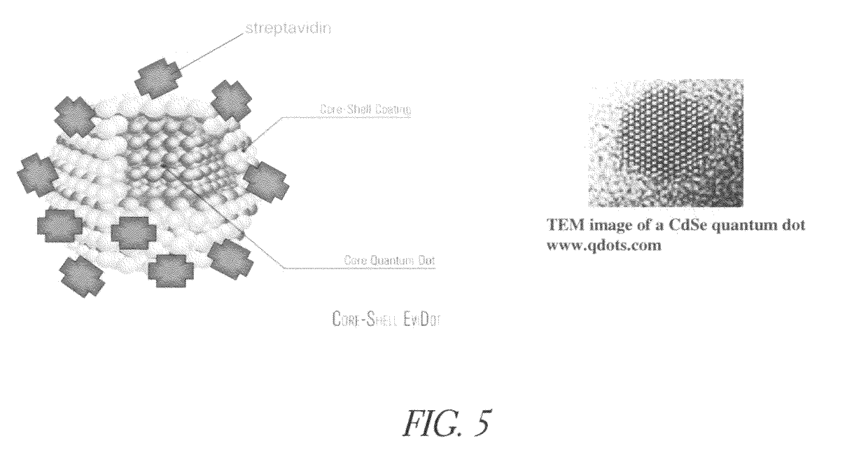

FIG. 6. Structure of the T4 phage. The capsid shell, head-tail connector, tail, and tail fibers are shown schematically. The diffraction pattern from polyheads showing a hexamer capsid unit has been fit onto the surface of the icosahedral particle (diameter approx. 55 mm). The monomer units are in gray. In our study, we used T7 phage, instead.

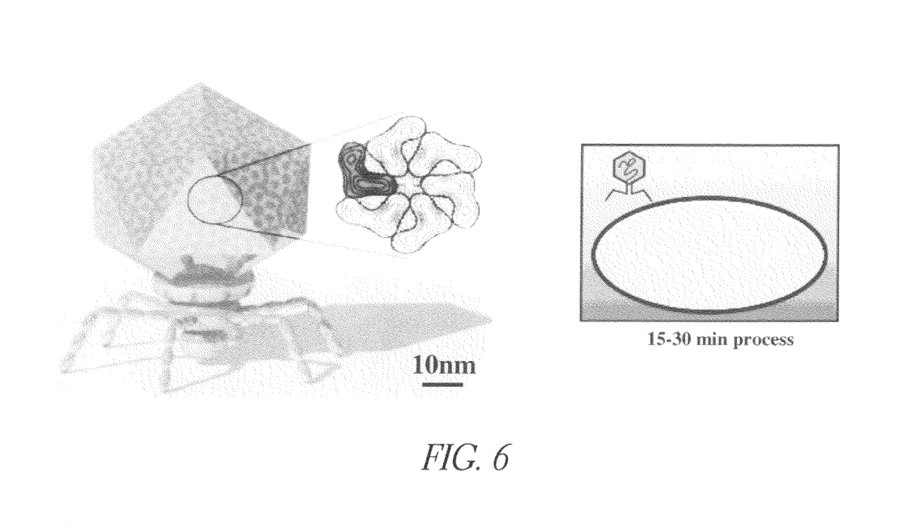

FIG. 7. Transfection of the phage to express biotin ligases on the capsid surface. The T7 capsid gene (gene 10) is located at about position 60 in the T7 genome, within the region of genes coding for proteins involved in the structure and assembly of T7. Capsid protein expression during infection is controlled by a promoter (φ10) and terminator (Tφ) for T7 RNA polymerase, and by string translation initiation signals (s10). The capsid protein is normally made in two forms, 10A (344 aa) and 10B (397 aa), related by a translational frameshift at 10A aa 341. The T7Select415 and T7Select1 vectors contain a multiple cloning site following aa 348 of a 10B gene that is in a single reading frame, i.e., only the truncated 10B form is made from these vectors. Expression of the capsid protein assembly from T7Select415 vectors is controlled as in the wild-type phage.

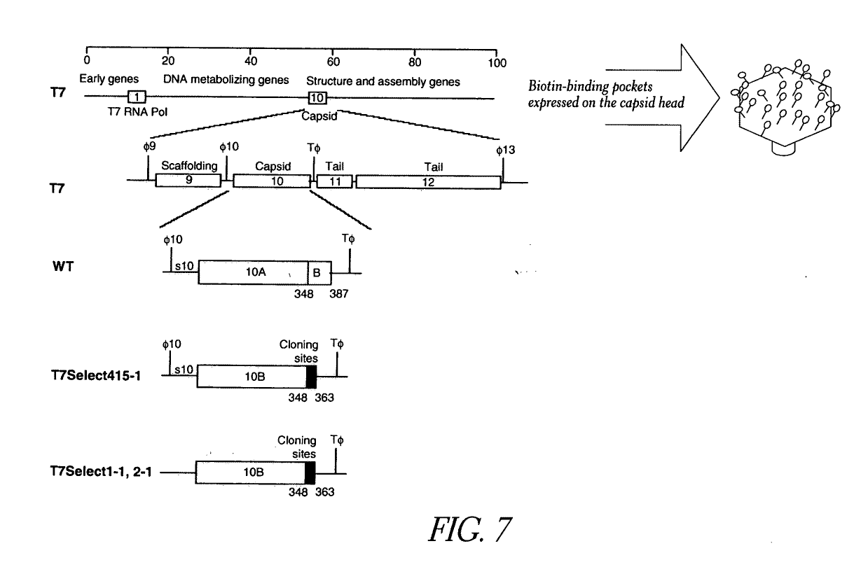

FIG. 8. Biotin protein ligase (BPL) on the capsid surface. The functionality of BPL is highly conserved through indigenous biological process; BPL will biotinylate biotin enzymes that are derived from divergent species including E. coli. BirA, the BPL of Escherichia coli biotinylates only a single cellular protein, Biotin Carboxyl Carrier Protein (BCCP), a subunit of acetyl-CoA carboxylase (the enzyme catalyzing the first committed step of fatty acid synthesis).

FIG. 9. High-throughput and high-sensitivity detection of phage using bioconjugated nanocrystals.

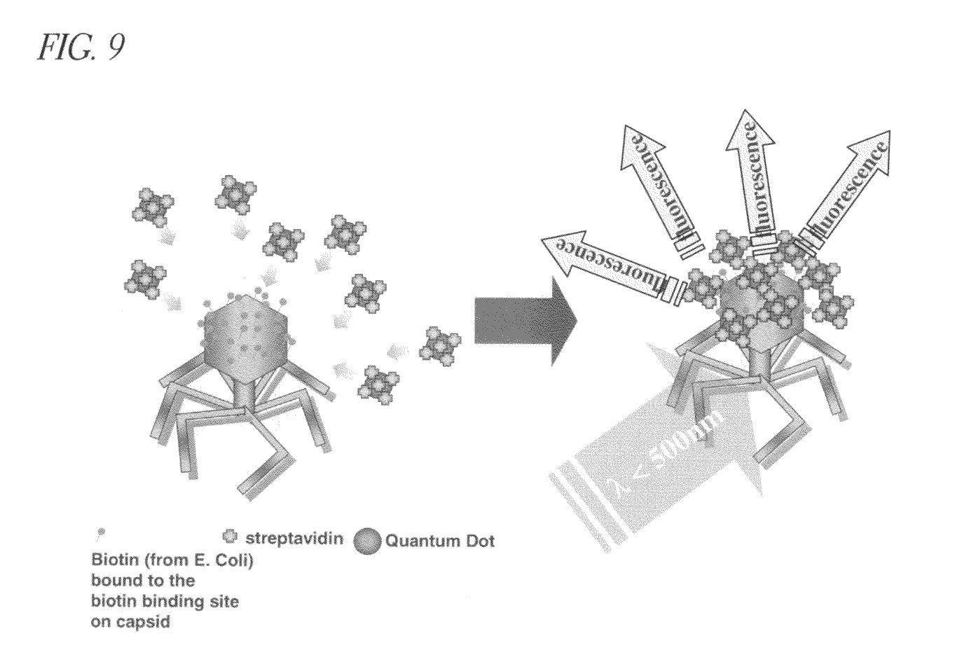

FIG. 10. The strategy of bacteria detection using quantum dot-conjugated phage.

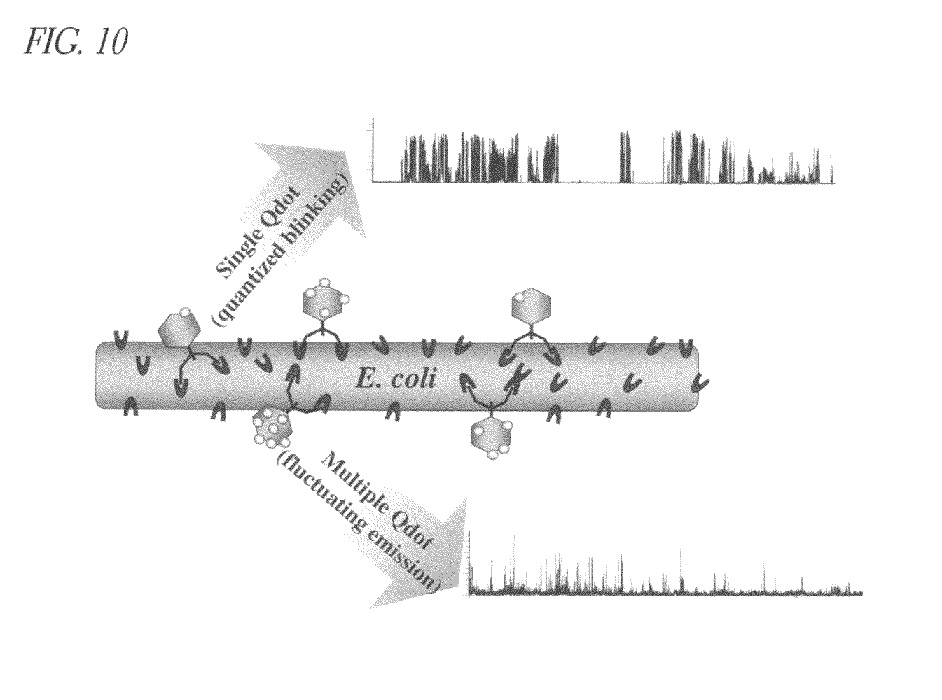

FIG. 11. Electron microscopy of quantum dot conjugated phage.

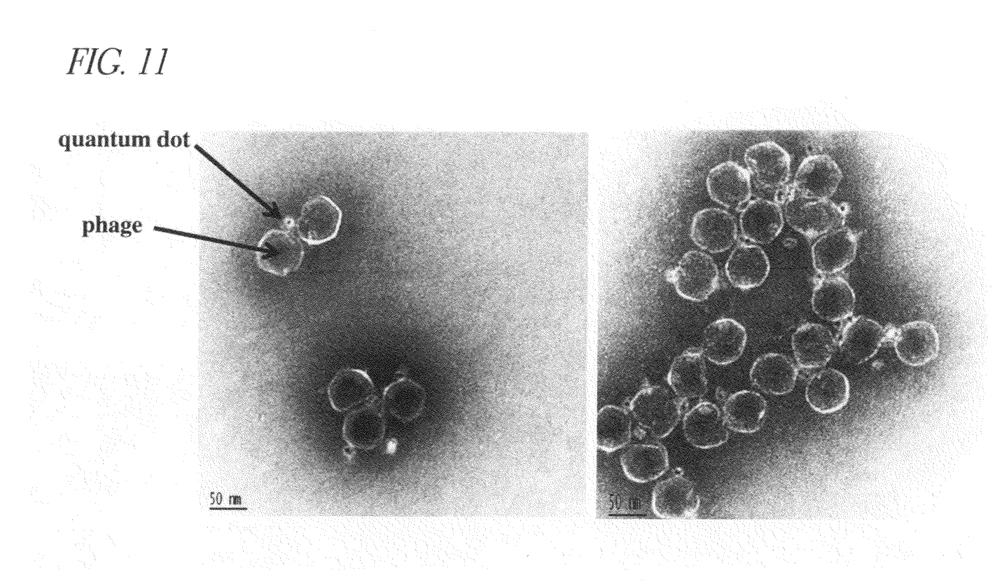

FIG. 12. QD concentration varied for quantitatively measuring the number of biotin binding sites on the capsid protein assembly.

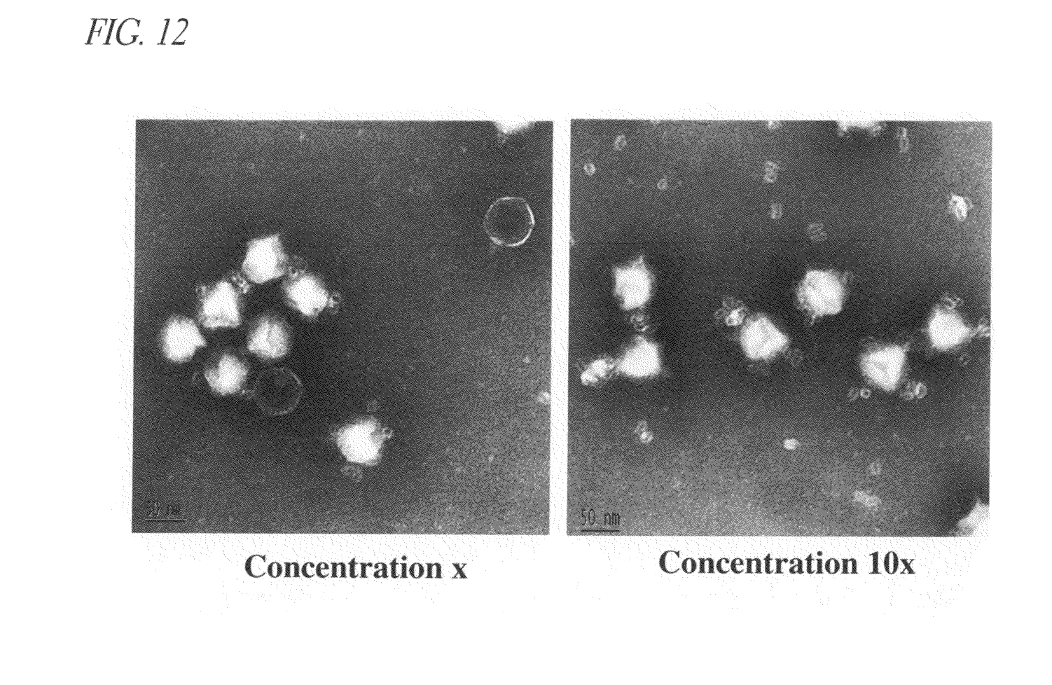

FIG. 13. QD concentration varied for quantitatively measuring the number of biotin binding sites on the capsid protein assembly (cont'd).

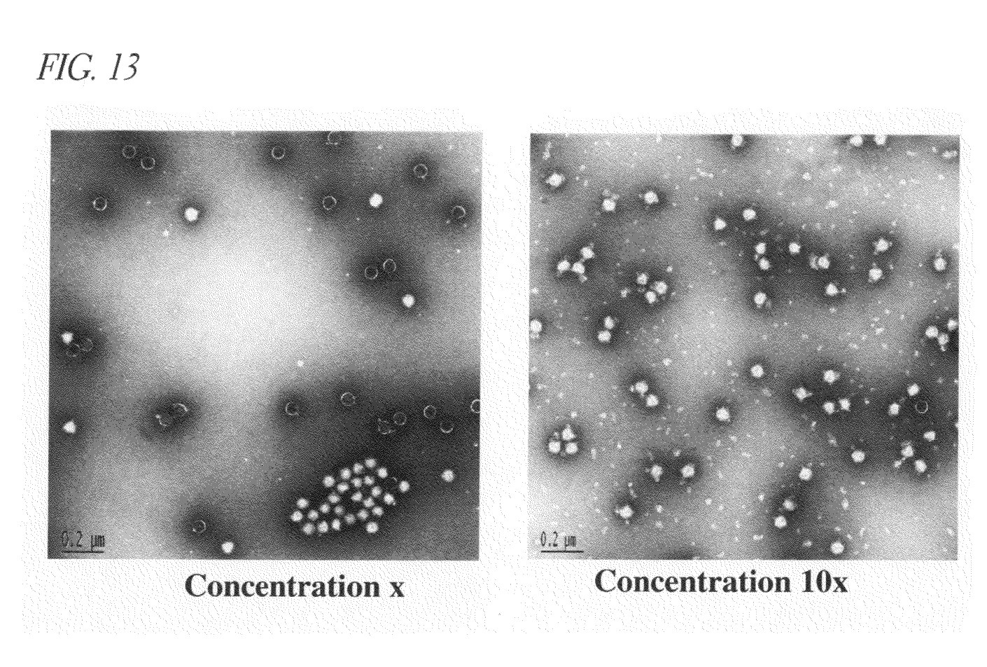

FIG. 14. Electron microscopy of Phage-QD targeting E. coli.

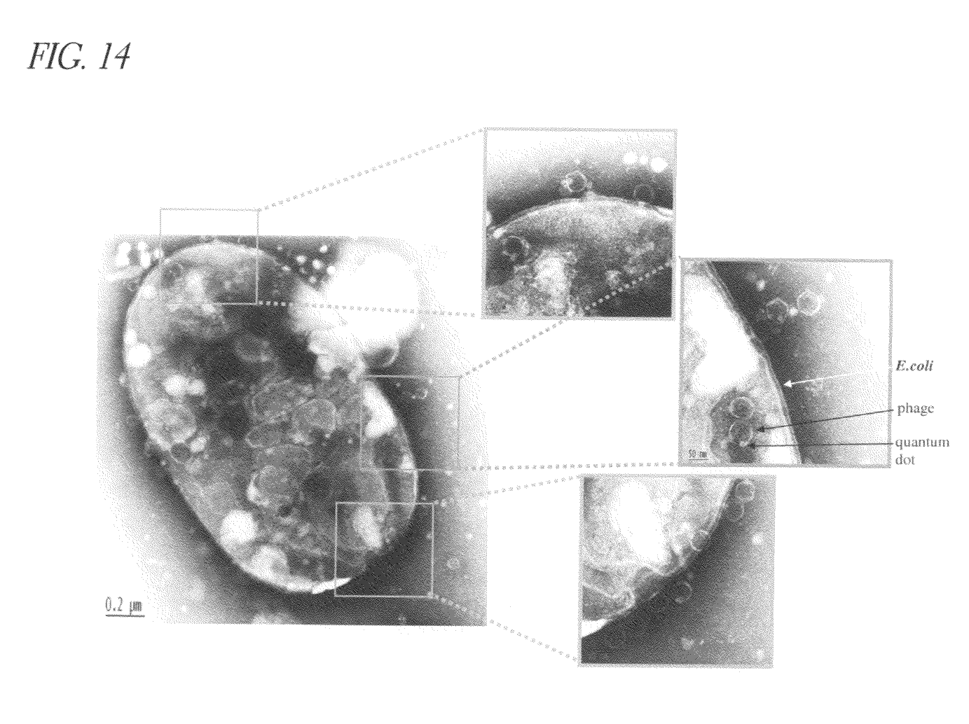

FIG. 15. Non-bleaching fluorescence signal.

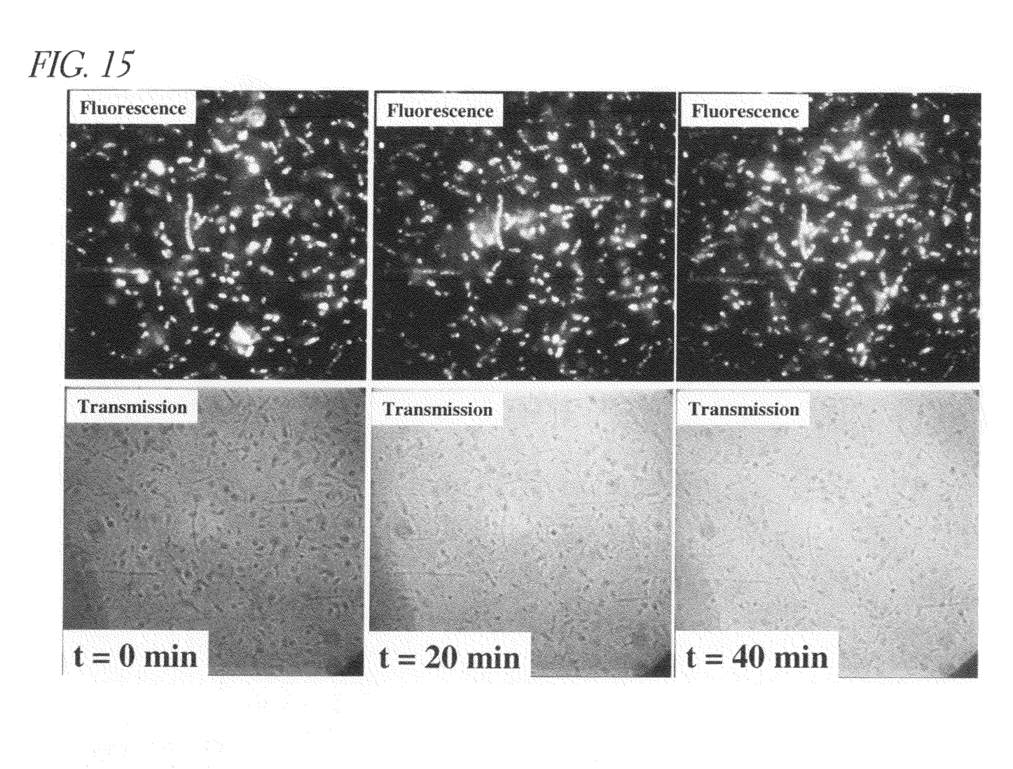

FIG. 16. Control Experiments without phage and with wild-type phage.

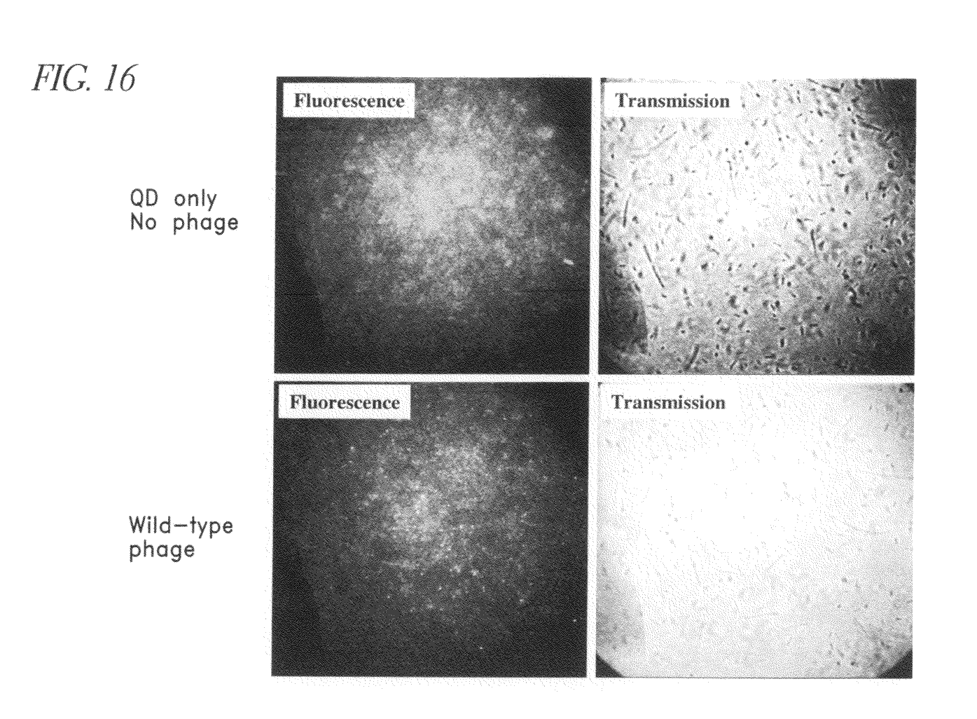

FIG. 17. Surface-immobilized bacteria on a hydrogel coated substrate.

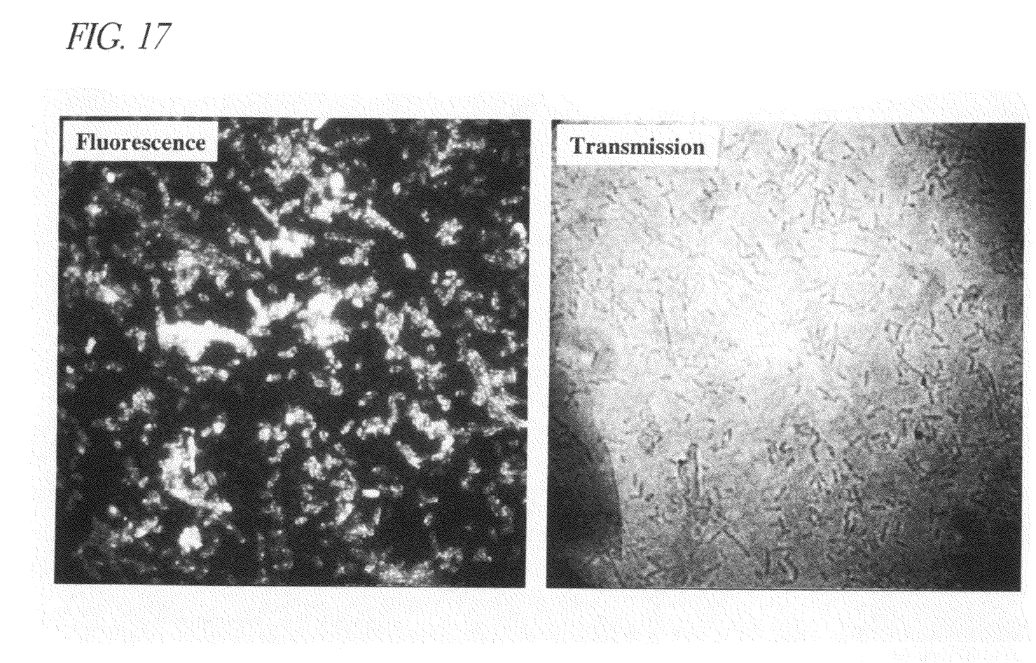

FIG. 18. Phage-QD complexes and analysis of quantized levels of QDs in complexes binding to bacteria.

### DETAILED DESCRIPTION OF THE PREFERRED EMBODIMENT

With current concerns of antibiotic resistant bacteria and bioterrorism, it has become important to rapidly identify infectious bacteria. Traditional technologies are often time consuming, involving isolation and amplification of the causative agents. Rapid and simple methods that can be employed to detect any desired target bacteria would be of great utility. We report a new, rapid and simple method that combines in vivo biotinylation of engineered host-specific bacteriophage and conjugation of the phage to streptavidin-coated quantum dots. The method provides detection and identification of as few as 10 bacterial cells/ml in experimental samples, including about one hundred fold amplification of the signal in one hour. We believe that the method can be applied to any bacteria susceptible to specific phages and would be particularly useful for detection of bacterial strains that are slow growing, e.g., Mycobacterium, or are highly infectious, e.g., Bacillus anthracis. We also envision simultaneous detection of different bacterial species in the sample as well as for applications in studying phage biology.

### Methods of Detection

The preferred embodiment centers on combining quantum dots (QDs) with engineered phage for the specific identification of biological targets including bacterial strain(s) or cells from clinical or environmental isolates. The novel combination of phage quantitatively labeled with QDs will enable the detection and quantification of low abundance targets present down to the single copy level. This preferred embodiment includes the following two aspects, (1) the method to combine phage-quantum dot complex, and (2) the idea to use this complex to target biological samples including bacteria strain(s).

In a time of bio-terrorism threats it is necessary to have new methods available for specifically detecting biological samples such as bacterial pathogens. Different challenges need to be addressed when trying to identify pathogenic bacteria in the ambient, non-laboratory situation. A detection system needs to be rapid, highly sensitive, and specific. The preferred embodiment centers on combining quantum dots (QDs) with engineered bacteriophage for the specific identification of target biological sample(s) including bacterial strain(s) from clinical or environmental isolates.

The unique optical characteristics of QDs such as photostability, size-dependent spectral properties on the same nanometer scale as its linked bacteriophage makes the combination highly suitable for imaging and discovery of single pathogenic bacteria. The combined system has the potential to overcome current limitations of conventional fluorophore-based methods to detect pathogenic bacteria. Current detections utilize optical and electrochemical measurement of nucleic acid or peptide sequences bound to organic fluorophore dyes. The current organic dyes have significant limitations including (1) photobleaching and (2) narrow excitation and spectral overlap in multiplexed detection. For a recent review see Gao X., Yang L., Petros A. J., Marshall F. F., Simons W. J., and Nie S., 2005, Current Opinion in Biotechnology 16:1-10.

The preferred embodiment combines functionalized (e.g., surface-coated with specific proteins, peptides, etc.) QDs with modified bacteriophage engineered to express surface-coat-molecule(s) capable of specifically binding to the functionalized QD. The modified bacteriophage are also adapted to be highly specific to target biological samples, for instance, strains of bacteria. The novel combination of phage quantitatively labeled with QDs will enable the detection and quantification of low abundance targets present down to the single copy level. Unlike organic fluorescence dyes, QDs are stable, non-diluting, non-bleaching, and they are fluorescence emitters covalently and quantitatively linked to phage.

This preferred embodiment includes the following two aspects, (1) the method to combine phage-quantum dot complex (phage-QD) and (2) the idea to use this complex to target bacteria strain(s). The procedure to practice the preferred embodiment is summarized as follows:

(1) Screen, select, and harvest specific phage targeting a specific target including bacterial strain(s) using standard biomolecular methods.

(2) Engineer the harvested phage to have the major head, capsid protein assembly of the phage express the binding sites of specific molecule(s) such as biotin ligase.

(3) Functionalize QDs with specific moiety such as streptavidin molecules capable of recognizing/binding the molecule(s) expressed on the engineered phage.

(4) The isolated and engineered phage (described in (1) and (2)) binds selectively to a target receptor on the biological target such as bacteria. The functionalized QDs (described in (3)) then covalently and quantitatively bind to the phage.

(5) The highly specific linkage of QD to the engineered phage and to the biological target such as bacteria (QD-phage-bacteria complex) provides a unique fluorescent signal at the single copy sensitivity.

(6) Single-color or multi-color, multiplexed detections of a variety of biological targets including bacterial strains are done using different QD-phage complexes. Such detections include high-throughput screening of low abundance bacterial strains using one or more of the methods involving microscopy, spectroscopy, and fluorescence flow cytometry. The preferred embodiment is amenable to applications using portable hand-held instruments.

Currently most advanced techniques to detect bacteria rely on the labeling of targets with green fluorescence protein (GFP) gene. For instance Oda et al. used GFP genes to express and fluorescently detect E. coli (M. Oda et al. 2004 Applied and Environmental Microbiology 70:527-534). This procedure requires laboratory equipment and expertise in molecular biology techniques. In addition, the difficulty in detecting bacteria with this approach occurs when the expression of GFP is low, requiring high cost and high-sensitivity fluorescence detection techniques such as single molecule imaging. Furthermore, the GFP photobleaches rapidly, allowing fluorescence measurement only a few seconds to a minute at ordinary microscopy conditions. When the minimum number of phage per bacteria to cause infection (multiplicity of infection, (M.O.I.)) is only a few, bacterial detection with GFP expression will be very difficult due to low fluorescence signal and fast photobleaching rate of GFP. However, the new method will provide the following advantages over the competing method relying on GFP-expression.

(1) Stable and economical optical detection of biological targets such as bacteria strains: Not only are QDs resistant to photobleaching, allowing for extended observation periods, but also QDs are up to 20 times brighter than traditional organic fluorophores, a result of high quantum yield and a large molar extinction coefficient. Our preferred embodiment, based on QDs will enable the detection of phage-bacterium interaction at the single copy level without the tedious efforts such as preparation of ultra-clean substrates and extreme rejection of background fluorescence signal. The target detection protocol is so simple that it can be done at a non-laboratory situation at a very low cost.

(2) Identifying multiple biological targets: The narrow emission band (the typical full width half maximum of 20 nm) allows for high spectral resolution. With the wide selection of emission wavelengths and the high spectral resolution, multiplexed experiments with various Phage-QDs are possible. QDs additionally have broad excitation spectra. This allows for the concurrent excitation of various QDs with a single excitation source. Meanwhile, the series of phage species may be selected or designed to target specific biological targets. These specific phage species can be combined with QDs of certain sizes having distinct colors to enable multiplexed detection using binding specificity between the phage and the targets.

Our preferred embodiment proves that this method will be of immediate use for the detection of a variety of biological targets such as bacterial strains, tumor cells, and other biomimetic targets. Such detections include high-throughput screening of low abundance bacterial strains using one or more of the methods involving microscopy, spectroscopy, and fluorescence flow cytometry.

This detection method can also be adapted to on-site detection of biological targets such as deadly pathogenic bacteria such as O157:H7 E. coli which occasionally causes massive meat product recalls, human illness and death. In one recent outbreak, 18 million pounds of meat were recalled because there was no high sensitivity detection method available. We also envision a generic method for quantitative detection of deadly pathogenic bacteria for the potential economic benefit of U.S. dairy and meat industries as well as bio-threat agent detection at a very low cost.

### DEFINITIONS

Unless defined otherwise, technical and scientific terms used herein have the same meaning as commonly understood by one of ordinary skill in the art to which this invention belongs. See, e.g., Singleton P and Sainsbury D., in Dictionary of Microbiology and Molecular Biology 3rd ed., J. Wiley & Sons, Chichester, N.Y., 2001; Madigan M. T. Martinko J. M. and Parker J. in Biology of Microorganisms, 9^{th} ed., Prentice-Hall, Inc., Upper Saddle River, N.J., 2000, and Fields Virology 4th ed., Knipe D. M. and Howley P. M. eds, Lippincott Williams & Wilkins, Philadelphia 2001.

The following compositions and methods offer an important improvement to existing methods for the detection and quantification of bacterial cells, including food pathogens, such as Listeria, E. coli, Salmonella, and Campylobacter, and medical pathogens, such as Bordetella pertussis, Chlamydia pneumoniae, and Mycoplasma pneumoniae.

The methods of the present invention provide high detection sensitivity in a short time without the need for traditional biological enrichment. For example, the present methods can provide for the detection or quantification of less than about 100, less than about 50 or less than about 10 bacterial cells in a sample. The present methods can provide for the detection or quantification of less than about 5, less than about 4, less than about 3, or less than about 2 bacterial cells in a sample. The methods of the present invention can provide for the detection and quantification of a single bacterial cell in a sample.

The methods of the present invention allow for the rapid detection and quantification of bacterial cells. For example, the methods of the present invention can be performed in less than about ten hours to less than about twelve hours, in less than about four hours to less than about three hours, and in about two hours or less.

The methods of the present invention can accommodate a wide range of samples sizes. For example, samples as large as about 25 grams (gm) or about 25 milliliters (ml) may be used. Samples of about 1 gram (gm) or about 1 ml or less may be used. If necessary, prior to an assay, samples may be concentrated to reduce the sample volume.

### Bacterial Cells

Any bacterial cell for which a bacteriophage that is specific for the particular bacterial cell has been identified can be detected by the methods of the present invention. Those skilled in the art will appreciate that there is no limit to the application of the present methods other than the availability of the necessary specific phage/target bacteria. Bacterial cells detectable by the present invention include, but are not limited to, bacterial cells that are food pathogens. Bacterial cells detectable by the present invention include, but are not limited to, all species of Salmonella, all species of E. coli, including, but not limited to E. coli O157:H7, all species of Listeria, including, but not limited to L. monocytogenes, and all species of Campylobacter. Bacterial cells detectable by the present invention include, but are not limited to, bacterial cells that are pathogens of medical or veterinary significance. Such pathogens include, but are not limited to, Bacillus spp., Bordetella pertussis, Campylobacter jejuni, Chlamydia pneumoniae, Clostridium perfringens, Enterobacter spp., Klebsiella pneumoniae, Mycoplasma pneumoniae, Salmonella typhi, Staphylococcus aureus, and Streptococcus spp. Cultures of all bacterial cells can be obtained, for example, from American Type Culture Collection (ATCC, P.O. Box 1549, Manassas, Va., USA). Bacterial cells detectable by the present invention also include, but are not limited to, contaminating bacterial cells found in systems of commercial significance, such as those used in commercial fermentation industries, ethanol production, antibiotic production, wine production, etc. Such pathogens include, but are not limited to, Lactobacillus spp. and Acetobacter spp. during ethanol production. Other examples of bacteria include those listed in W. Levinson et al., Medical Microbiology & Immunology, McGraw-Hill Cos., Inc., 6th Ed., pages 414-433 (2000). All bacterial cultures are grown using procedures well known in the art.

The range of bacterial cells to be detected is limited only by host ranges of available bacteriophages. Of particular interest are pathogenic bacteria which are capable of contaminating food and water supplies and are responsible for causing diseases in animals and man. Such pathogenic bacteria will usually be gram-negative, although the detection and identification of gram-positive bacteria is also a part of the present invention. A representative list of bacterial hosts of particular interest (with the diseases caused by such bacterial hosts) includes Actinomyces israelii (infection), Aeromonas hydrophila (gastroenteritis, septicemia), Bacillus anthracis (Anthrax: cutaneous, pulmonary), Bacillus subtilis (not considered pathogenic or toxigenic to humans, animals, or plants), Bacteriodes caccae (anaerobic infection), Bacteriodes distasonis (anaerobic infection), Bacteriodes merdae (anaerobic infection), Bacteriodes ovatus (anaerobic infection), Bacteriodes vulgatus (anaerobic infection), Bacteroides fragilis (anaerobic infection), Bacteroides thetaiotaomicron (anaerobic infection), Bordetella pertussis (Whooping cough), Borrelia burgdorferi (Lyme Disease), Brucella abortus (Brucellosis-cattle), Brucella canis (Brucellosis-dogs), Brucella melitensis (Brucellosis-sheep and goats), Brucella suis (Brucellosis-hogs), Burkholderia pseudomallei (infection: acute pulmonary, disseminated septicemic, nondisseminated septicemic, localized chronic suppurative), Campylobacter coli (diarrhea), Campylobacter fetus (bacteremia), Campylobacter jejuni (fever, abdominal cramps, and diarrhea, Guillain-Barre syndrome), Chlamydia trachomatis (Chlamydia), Clostridium botulinum (botulism), Clostridium butyricum (neonatal necrotizing enterocolitis, NEC), Clostridium difficile (NEC), Clostridium perfringens (myonecrosis-gas gangrene), clostridial cellulites, clostridial myositis, food disease, NEC), Clostridium tetani (tetanus), Corynebacterium diphtheriae (diptheria), Enterococcus durans (infection), Enterococcus faecalis (nosocomial infection), Enterococcus faecium (nosocomial infection), Erysipelothrix rhusiopathiae (erysipelothricosis), Escherichia coli (inflammatory or bloody diarrhea, urinary infection, bacteremia, meningitis), Francisella tularensis (tularemia), the genus Fusobacterium (anaerobic infection), Haemophilus aegyptius (mucopurulent conjunctivitis, bacteremic Brazilian purpuric fever), Haemophilus aphrophilus (bacteremia, endocarditis and brain abscess), Haemophilus ducreyi (chancroid venereal disease), Haemophilus influenzae (bacterial meningitis, bacteremia, septic arthritis, pneumonia, tracheobronchitis, otitis media, conjunctivitis, sinusitis, acute epiglottitis, endocarditis), Heaemophilus parainfluenzae (bacteremia, endocarditis and brain abscess), Helicobacter pylori (gastric and duodenal ulcers, gastric cancers), Klebsiella pneumoniae (respiratory, urinary infection), Legionella pneumonphila (Legionaire's disease), the genus Leptospira (leptospirosis, or infectious spirochetal jaundice), Listeria ivanovii (listeriosis), Listeria monocytogenes (listeriosis), Listeria seeligeri (listeriosis), Morganella morganii (infection), Mycobacterium africanum (tuberculosis), Mycobacterium avium-intracellulare (Lady Windermere syndrome, mycobacterium avium complex, MAC), Mycobacterium bovis (tuberculosis), Mycobacterium chelonei (infection), Mycobacterium fortuitum (infection), Mycobacterium kansasii (infection), Mycobacterium leprae (leprosy), Mycobacterium marinum (infection), Mycobacterium tuberculosis (tuberculosis), Mycobacterium ulcerans (infection), Mycobacterium xenopi (infection), Neisseria gonorrhoeae (gonorrhea), Neisseria meningitidis (meningitis), Nocardia asteroids (nocardiosis), Prevotella melaminogenica (anaerobic infection), Proteus mirabilis (infection), Proteus mysofaciens (infection), Proteus vulgaris (infection), Providencia alcalifaciens (infection), Providencia rettgeri (infection), Providencia stuartii (infection), Pseudomonas acidovorans (nosocomial infection), Pseudomonas aeruginosa (nosocomial infection, i.e. in cystic fibrosis patients, burn victims, patients with permanent catheters), Pseudomonas fluorescens (nosocomial infection), Pseudomonas paucimobilis (nosocomial infection), Psuedomonas putida (nosocomial infection), Rickettsia rickettsii (Rocky Mountain spotted fever), Salmonella anatum (gastroenteritis, septicemia), Salmonella bovismorbficans (gastroenteritis, septicemia), Salmonella choleraesuis (gastroenteritis, septicemia), Salmonella Dublin (gastroenteritis, septicemia), Salmonella enteritidis (gastroenteritis, septicemia, enteric fever, bacteremia), Salmonella hirschfeldii (enteric fever), Salmonella Newington (gastroenteritis, septicemia), Salmonella paratyphi (paratyphoid), Salmonella schottmulleri (gastroenteritis, septicemia), Salmonella shottmuelleri (enteric fever), Salmonella typhi (typhoid fever), Serratia marcescens (wound infections), Shigella boydii (shigellosis), Shigella dysenteriae (shigellosis), Shigella flexneri (shigellosis), Shigella sonnei (shigellosis), Spirillum minus (rat-bite fever), Staphylococcus aureus (infections, food poisoning, toxic shock syndrome, pneumonia, bacteremia, endocarditis osteomyelitis enterocolitis, subcutaneous abscesses, exfoliation, meningitis), Streptobacillus moniliformis (rat-bite fever), Streptococcus agalactiae (neonatal sepsis, postpartum sepsis, endocarditis, and septic arthritis), Streptococcus antinosus (invasive infections), Streptococcus bovis (bacterial endocarditis), Streptococcus constellatus (invasive infections), Streptococcus iniae (cellulitis and invasive infections), Streptococcus intermedius (invasive infections), Streptococcus mitior (bacterial endocarditis), Streptococcus nutans (endocarditis), Streptococcus pneumoniae (pneumonia, acute otitis media, infection of the paranasal sinuses, acute purulent meningitis, bacteremia, pneumococcal endocarditis, pneumococcal arthritis, pneumococcal peritonitis), Streptococcus pyogenes (pharyngitis, tonsillitis, wound and skin infections, septicemia, scarlet fever, pneumonia, rheumatic fever and glomerulonephritis), Streptococcus salivarius (bacterial endocarditis), Streptococcus sanguis (bacterial endocarditis), Treponema palladuni (syphilis), Vibrio alginolyticus (diarrhea, infection), Vibrio cholerae (cholera), Vibrio hollisae (diarrhea, infection), Vibrio mimicus (diarrhea, infection), Vibrio parahaemolyticus (diarrhea, infection), Vibrio vulnificus (diarrhea, infection), and Yersinia pestis (plague). The invention may also be used to detect subspecies of bacteria, for example E. coli O157:H7.

In February 2002, the National Institute of Allergy and Infectious Diseases (NIAID) convened the first Blue Ribbon Panel on Bioterrorism and Its Implications for Biomedical Research. This panel of experts was brought together by NIAID to provide objective expertise on the Institute's future counter-bioterrorism research agenda for anthrax, smallpox, botulism, plague, tularemia, and viral hemorrhagic fevers, the pathogens commonly referred to as CDC Category A agents (Table 1). On Oct. 22 and 23, 2002, the NIAID convened another Blue Ribbon Panel of experts to provide objective expertise on the Institute's future biodefense research agenda, as it relates to the NIAID Category B and C Priority Pathogens (Table 1). As a result of these meetings and deliberations a research agenda was developed and widely distributed to the scientific community. One of the goals in managing pathogenic bacteria is to develop improved diagnostics. The initial clinical signs and symptoms of many agents considered biothreats are nonspecific and resemble those of common infections. Therefore, the ability to rapidly identify the introduction of a bioterrorism bacteria or toxin will require diagnostic tools that are highly sensitive, specific, inexpensive, easy to use, and located in primary care settings. Environmental detection is also an important aspect of disease prevention and control.

TABLE 1
NIAID Category A, B, and C Priority Pathogenic Bacterial

| Category | Pathogenic Bacteria |
|---|---|
| A | Bacillus anthracis (anthrax) |
| | Clostridium botulinum (botulism) |
| | Yersinia pestis (plague) |
| | Francisella tularensis (tularemia) |
| B | Burkholderia pseudomallei (melioidosis) |
| | Coxiella burnetii (Q fever) |
| | Brucella species (brucellosis) |
| | Burkholderia mallei (glanders) |
| | Epsilon toxin (of Clostridium perfringens) |
| | Staphylococcal enterotoxin B |
| | Typhus fever (Rickettsia prowazekii) |
| | Food- and Water-borne Pathogens |
| | Diarrheagenic Escherichia coli |
| | Pathogenic Vibrios |
| | Shigella species |
| | Salmonella species |
| | Listeria monocytogenes |
| | Campylobacter jejuni |
| | Yersinia enterocolitica |
| C | Multi-drug resistant TB |

### Bacteriophage

Bacteriophage, also called phage, are highly selective for their hosts. Bacteriophage typing is useful at the species and strain level for identifying bacteria, for instance, in epidemiological investigation of food-borne illness. The specificity of a phage for its host is determined at two levels. Each phage has a host receptor that for tailed phage typically recognizes elements of the phage baseplate and phage tail fibers. Interaction of these components with complementary elements on the bacterial cell surface determines the ability of the phage to bind to the cell and inject its DNA. Enzymatic activity of baseplate elements is sometimes but not always required. There is substantial evidence that phage breeding, genetic engineering of fiber elements, and hybridization, can alter phage specificity at this level. The second level of control over specificity is the events occurring within the bacterial cell, after injection of the phage DNA. Factors that can impact the phage's effectiveness include the presence of restriction enzyme systems in the host and the presence or absence of corresponding protective modifications of the phage DNA, the presence of immunity repressors, and the ability of phage promoters and accessory proteins to co-opt the host RNA polymerase to make phage proteins. Immunity repressors result from the presence of closely related integrated prophages in the target genome and are typically of narrow specificity. Restriction systems and promoter specificity have similar effects on phage expression and plasmid expression, the latter being fairly well understood.

Besides exhibiting specificity, phages have the ability to produce a substantial amplification in a short time. Under optimum infection and host growth medium conditions, a given phage/bacterium combination gives rise to a consistent number of phage progeny. Generally, the lytic infection cycle produces 100 or more progeny phage particles from a single infected cell in about one hour. However, there are exceptions. For example, phi29 of B. subtilis is a premier phage system for study of morphogenesis because it gives a burst of 1,000 in a 35-minute life cycle. Bacteria can be multiply infected by phages (multiplicity of infection, m.o.i.), and the phage “burst” (progeny produced per cell) depends on the multiplicity. To produce high yields an m.o.i. of 10 is generally used. Within an assay it may be necessary to include control comparison standards, done in the same medium, with known numbers of phages infecting known numbers of substrate-bound target cells.

For the detection of a given bacterial cell, a bacteriophage that is capable of infecting the bacterial cell, replicating within the bacterial cell and lysing the bacterial cell is selected. For any given bacterial cell a wide variety of bacteriophages are available, for example, from ATCC or by isolation from natural sources that harbor the host cells. The bacteriophage should also exhibit specificity for the bacterial cell. A bacteriophage is specific for a bacterial cell when it infects the given bacterial cell and does not infect bacterial cells of other species or strains. For the detection of a particular bacterial cell, one would also preferably select a bacteriophage that gives an optimal or maximal burst size.

The range of bacterial cells that can be detected by the present invention is limited only by the availability of a bacteriophage specific for the bacterial cell and will be realized to be vast by those skilled in the art. For example, a searchable database of bacteriophage types available from ATCC is on the worldwide web at atcc.org. Other such depositories also publish equivalent data in their catalogues, and this may be used to identify possible bacteriophage reagents for the methods of the present invention.

Examples of specific bacteria/bacteriophage pairings include PP01, which is specific for E. coli O157:H7 (see, Oda M. et al. 2004 Appl Envir Microbial 70:527-534); phiA1122, which is specific for Yersinia Pestis (see, Garcia E. et al. 2003 J Bacterial 185:5248-5262); D29, which is specific for Mycobacterium tuberculosis (see, McNerney R. et al. 2004 J Clin Microbial 42:2115-2120); T4, which is specific for E. coli (Molecular Biology of Bacteriophage T4, ed. Karam, J. D. (Am. Soc. Microbiol., Washington, D.C.)); and Listeria monocytogenes phage A511, which is specific for L. monocytogenes (see, Loessner et al. 1996 Appl and Environ Microbial 62:1133-1140). Over fourteen different Campylobacter phages are available from ATCC. A number of these are specific for C. jejuni and C. coli and form the basis for a bacteriophage typing system (Grajewski B A et al. 1985 J Clin Microbial 22:13-18). ATCC lists over twenty-four different phages specific for Salmonella; included is phi29, a well-studied phage for Salmonella typhimurium (Zinder, N. D. and Lederberg, J. 1952 J Bacteriology 64:679-699).

High titer bacteriophage stocks are produced on an appropriate host cell strain by procedures well known in the art. For example, plate or broth lysis methods may be used in the production of high titer stocks of bacteriophage. The culture of many other bacteria/bacteriophage pairings is well known to those of skill in the art. See, for example, U.S. Pat. Nos. 5,679,510; 5,914,240; 5,985,596; 5,958,675; 6,090,541; and 6,355,445. See also, for example, Bacteriophages, Mark Adams, InterSciences Publishers, Inc., New York, (1959).

### Samples

Samples include, but are not limited to, environmental or food samples and medical or veterinary samples. Samples may be liquid, solid, or semi-solid. Samples may be swabs of solid surfaces. Samples may include environmental materials, such as the water samples, or the filters from air samples or aerosol samples from cyclone collectors. Samples may be of meat, poultry, processed foods, milk, cheese, or other dairy products. Medical or veterinary samples include, but are not limited to, blood, sputum, cerebrospinal fluid, and fecal samples and different types of swabs.

Samples may be used directly in the detection methods of the present invention, without preparation or dilution. For example, liquid samples, including but not limited to, milk and juices, may be assayed directly. Samples may be diluted or suspended in solution, which may include, but is not limited to a buffered solution or a bacterial culture medium. A sample that is a solid or semi-solid may be suspended in a liquid by mincing, mixing or macerating the solid in the liquid. A sample should be maintained within a pH range that promotes bacteriophage attachment to the host bacterial cell. A sample should also contain the appropriate concentrations of divalent and monovalent cations, including but not limited to Na⁺, Mg⁺⁺, and K⁺. Preferably a sample is maintained at a temperature that maintains the viability of any pathogen cells contained within the sample.

### Biotinylation Domains

Biotin (vitamin H), an essential coenzyme synthesized by plants and most procaryotes, is required by all organisms. In cells, biotin in its physiologically active form is covalently attached at the active site of a class of important metabolic enzymes, the biotin carboxylase and decarboxylases.

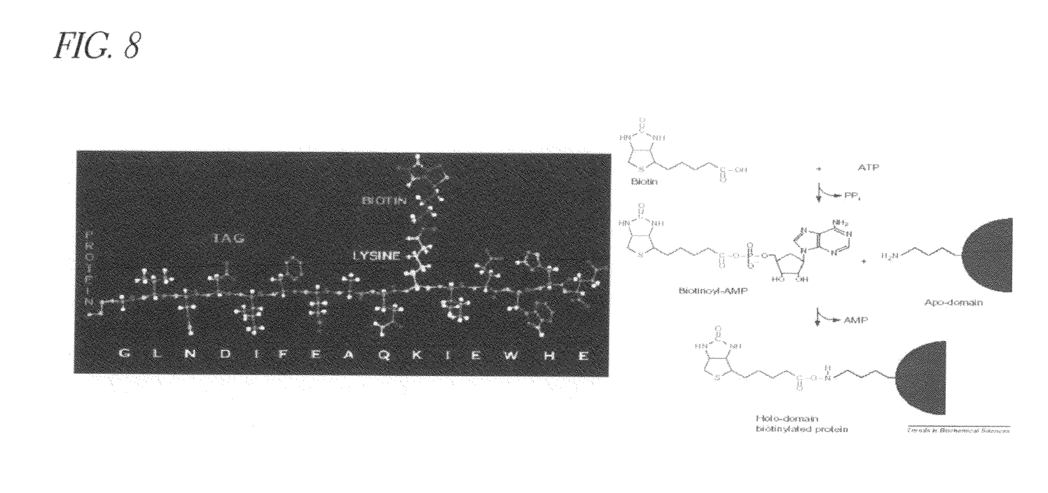

Biotin protein ligase (BPL), also known as holocarboxylase synthetase, is the enzyme responsible for the covalent attachment of biotin to the cognate proteins. Biotin is attached post-translationally by BPL via an amide linkage to a specific lysine residue of newly synthesized carboxylases in a two-step reaction (FIG. 8).

Although the occurrence of biotin-dependent enzymes is ubiquitous in nature, biotinylation is a relatively rare modification in the cell, with between one and five biotinylated protein species found in different organisms (Cronan J. E. Jr. 1990 J Biol Chem 265:10327-10333). Thus, biotin ligase catalyzes a reaction of stringent specificity. The functional interaction between BPL and its protein substrate shows a very high degree of conservation throughout evolution because biotinylation will occur when the two proteins come from widely divergent biological sources (Cronan J. E. Jr. 1990 J Biol Chem 265:10327-10333; Leon-Del-R10 et al. 1995 Proc Natl Acad Sci U.S.A. 92:4626-4630; MacAllister and Coon 1966 J Biol Chem 241:2855-2861; Tissot et al. 1996 Biochem J 314:391-395). The best characterized BPL is the multifunctional BirA protein from Escherichia coli.

Proteins that are biotinylated contain a biotinylation substrate sequence domain that is biotinylated by the BPL. It was found that fusion of a biotinylation domain from a naturally biotinylated protein with any protein of interest provided a general method of specifically labeling chimeric proteins with biotin at a single site. Screens of four biased peptide libraries identified a “consensus peptide” for biotinylation. The “consensus sequence” does not represent absolute sequence requirements for biotinylation, but merely a consensus of the peptide libraries screened. The identified sequences, when fused to either the N- or C-terminus of a variety of proteins can be biotinylated either in vitro or in vivo.

A summary of amino acid sequences active in biotin holoenzyme synthetase (BHS)-catalyzed biotinylation of peptide substrates is described by the consensus sequence: L-X₂—X₃—I—X₅—X₅—X₇—X₈—K—X₁₀—X₁₁—X₁₂—X₁₃ (SEQ ID NO: 1), where X₂ is any amino acid; X₃ is any amino acid except L, V, I, W, F, or Y; X₅ is F or L; X₆ is E or D; X₇ is A, G, S, or T; X₈ is Q or M; X₁₀ is I, M, or V; X₁₁ is E, L, V, Y, or I; X₁₂ is W, Y, V, F, L or I; and X₁₃ is any amino acid except D or E.

A 23-residue peptide (MAGGLNDIFEAQKIEWHEDTGGS) (SEQ ID NO: 2) was identified, which, when fused to the N-terminus of maltose binding protein, was identical to the natural biotin carboxyl carrier protein (BCCP) substrate in the biotinylation reaction by BirA. On its own, this same peptide was also identical to the natural substrate in BirA-catalyzed modification. Measurements of biotinylation of a series of truncates of the 23-mer allowed identification of a 14-residue minimal substrate (GLNDIFEAQKIEWH) (SEQ ID NO: 3) that is biotinylated at a rate within two-fold of the natural protein substrate. Although this 14-mer works well, a slightly extended 15-mer, termed the AviTag, (GLNDIFEAQKIEWHE) (SEQ ID NO: 4) is consistently biotinylated at a rate slightly higher than that of the natural substrate.

Fusion of biotinylation domains to proteins works well at either the N terminus or the C terminus of a target protein. At the N-terminus, an ATG initiation codon is necessary, which, for expression in E. coli, may be followed by an Ala or Ser codon to confer proteolytic stability if the N-terminal Met is removed by methionine aminopeptidase. For fusion at the C terminus of a target protein, the biotinylation domain may be connected to the protein through a short Gly-Gly linker, although it is not clear that the linker is necessary. When a biotinylation domain such as AviTag is fused to a location on the protein other than the termini, the result will likely be extremely variable, depending on whether the biotinylation domain is folded in such a way that the biotin protein ligase (e.g., BirA) can recognize it as a substrate.

Cloning of Biotinylation Domain into Phage Capsid Proteins

In the methods of the invention, a nucleic acid sequence encoding a biotinylation domain is fused with the open reading frame of a bacteriophage capsid protein. It will be appreciated by those of skill in the art that, for a given bacteriophage, there may be multiple capsid proteins into which the biotinylation domain may be inserted so that when the capsid protein is biotinylated, progeny phage will display the biotin moiety such that it is accessible to a biotin-specific ligand (e.g., streptavidin).

To insert a biotinylation domain into a capsid protein by standard molecular cloning methods, it is helpful to know the nucleotide sequence of the bacteriophage capsid protein of interest. Table 2 lists bacteriophages, Genbank accession numbers for bacteriophage genomic sequences sequenced to date, exemplary capsid proteins, the amino acid lengths of the capsid proteins, and Genbank accession numbers for the capsid proteins.

TABLE 2
Bacteriophages and exemplary capsid proteins

| | | | | Accession |
|---|---|---|---|---|
| | Accession | | Length | No. (Capsid |
| Bacteriophage | No. (Genome) | Exemplary Capsid Protein | (aa) | Protein) |
| Acholeplasma phage L2 | L13696 | envelope protein | 738 | NP_040821 |
| Acholeplasma phage MV-L1 | X58839 | ND* | ND | ND |
| Acidianus filamentus virus 1 | AJ567472 | ND | ND | ND |
| Actinoplanes phage phiAsp2 | AY576796 | ND | ND | ND |
| Acyrthosiphon pisum bacteriophage APSE-1 | AF157835 | major head protein | 423 | NP_050985 |
| Aeromonas phage 31 | AY962392 | ND | ND | ND |
| Alteromonas phage PM2 | AF155037 | major capsid protein P2 | 269 | NP_049903 |
| Bacillus anthracis phage Cherry | DQ222851 | major capsid protein, HK97 family | 392 | YP_338137 |
| Bacillus anthracis phage Gamma | DQ222853 | major capsid protein, HK97 family | 392 | YP_338188 |
| Bacillus anthracis phage W Beta | DQ289555 | putative major capsid protein | 392 | YP_459969 |
| Bacillus clarkii bacteriophage BCJA1c | AY616446 | major capsid protein | 314 | YP_164418 |
| Bacillus phage GA-1 virion | X96987 | major head protein | 472 | NP_073691 |
| Bacillus phage phi29 | M11813 | major head protein | 448 | NP_040725 |
| Bacillus thuringiensis bacteriophage Bam35c | AY257527 | ND | ND | ND |
| Bacillus thuringiensis phage GIL16c | AY701338 | ND | ND | ND |
| Bacteriophage 11b | AJ842011 | major capsid protein precursor | 379 | YP_112497 |
| Bacteriophage 187 | AY954950 | ND | ND | ND |
| Bacteriophage 2638A | AY954954 | ND | ND | ND |
| Bacteriophage 29 | AY954964 | ND | ND | ND |
| Bacteriophage 37 | AY954958 | ND | ND | ND |
| Bacteriophage 3A | AY954956 | ND | ND | ND |
| Bacteriophage 42e | AY954955 | ND | ND | ND |
| Bacteriophage 44RR2.8t | AY375531 | major capsid protein; gp23 | 529 | NP_932516.1 |
| Bacteriophage 47 | AY954957 | ND | ND | ND |
| Bacteriophage 52A | AY954965 | ND | ND | ND |
| Bacteriophage 53 | AY954952 | ND | ND | ND |
| Bacteriophage 55 | AY954963 | ND | ND | ND |
| Bacteriophage 66 | AY954949 | ND | ND | ND |
| Bacteriophage 69 | AY954951 | ND | ND | ND |
| Bacteriophage 71 | AY954962 | ND | ND | ND |
| Bacteriophage 77 | AY508486 | ND | ND | ND |
| Bacteriophage 85 | AY954953 | ND | ND | ND |
| Bacteriophage 88 | AY954966 | ND | ND | ND |
| Bacteriophage 92 | AY954967 | ND | ND | ND |
| Bacteriophage 933W | AF125520 | ND | ND | ND |
| Bacteriophage 96 | AY954960 | ND | ND | ND |
| Bacteriophage A118 | AJ242593 | major capsid protein | 299 | NP_463467 |
| Bacteriophage AP205 | AF334111 | coat protein | 131 | NP_085472 |
| Bacteriophage Aaphi23 | AJ560763 | putative minor head protein | 800 | NP_852755 |
| Bacteriophage Aeh1 | AY266303 | gp23 major head protein | 534 | NP_944113 |
| Bacteriophage B103 | X99260 | major head protein | 449 | NP_690641 |
| Bacteriophage B3 | AF232233 | capsid protein | 309 | YP_164075 |
| Bacteriophage D3112 | AY394005 | putative major head subunit protein | 302 | NP_938242 |
| Bacteriophage EJ-1 | AJ609634 | major head protein | 330 | NP_945286 |
| Bacteriophage EW | AY954959 | ND | ND | ND |
| Bacteriophage Felix 01 | AF320576 | ND | ND | ND |
| Bacteriophage G1 | AY954969 | ND | ND | ND |
| Bacteriophage HK620 | AF335538 | capsid protein | 423 | NP_112079 |
| Bacteriophage HK97 | AF069529 | major head subunit precursor | 385 | NP_037701 |
| Bacteriophage IN93 | AB063393 | coat protein | 138 | NP_777330 |
| Bacteriophage JK06 | DQ121662 | hypothetical minor outer capsid | 124 | YP_277475 |
| | | protein | | |
| Bacteriophage K139 | AF125163 | putative major capsid protein | 341 | NP_536650 |
| Bacteriophage KS7 | AY730274 | ND | ND | ND |
| Bacteriophage KVP40 | AY283928 | head vertex protein | 298 | NP_899311 |
| Bacteriophage L-413C | AY251033 | ND | ND | ND |
| Bacteriophage L5 | L06183 | ND | ND | ND |
| Bacteriophage Lc-Nu | AY131267 | major head protein | 389 | YP_358764 |
| Bacteriophage Mx8 | AF396866 | ND | ND | ND |
| Bacteriophage N15 | AF064539 | ND | ND | ND |
| Bacteriophage P27 | AJ298298 | putative major capsid protein | 407 | NP_543092 |
| Bacteriophage P4 | X51522 | head size determination protein sid | 244 | NP_042042 |
| Bacteriophage PSP3 | AY135486 | ND | ND | ND |
| Bacteriophage PT1028 | AY954948 | ND | ND | ND |
| Bacteriophage PY54 | AJ564013 | capsid protein | 303 | NP_892049 |
| Bacteriophage RM 378 | AX059140 | similar to major head protein | 523 | NP_835728 |
| Bacteriophage ROSA | AY954961 | ND | ND | ND |
| Bacteriophage RTP | AM156909 | ND | ND | ND |
| Bacteriophage S-PM2 | AJ630128 | major capsid protein gp23 | 468 | YP_195142 |
| Bacteriophage SH1 | AY950802 | ND | ND | ND |
| Bacteriophage SPBc2 | AF020713 | ND | ND | ND |
| Bacteriophage SPP1 | X97918 | coat protein | 324 | NP_690674 |
| Bacteriophage T3 | AJ318471 | major capsid protein 10A | 347 | NP_523335 |
| Bacteriophage T5 | AY543070 | major head protein precursor | 458 | YP_006977 |
| Bacteriophage Tuc2009 | AF109874 | ND | ND | ND |
| Bacteriophage VSKK | AF452449 | putative major coat protein precursor | 82 | NP_536621 |
| Bacteriophage VT2-Sa | AP000363 | ND | ND | ND |
| Bacteriophage VWB | AY320035 | ND | ND | ND |
| Vibrio phage Vf33 | AB012573 | ND | ND | ND |
| Bacteriophage VfO3K6 | AB043678 | ND | ND | ND |
| Bacteriophage VfO4K68 | AB043679 | ND | ND | ND |
| Bacteriophage WPhi | AY135739 | ND | ND | ND |
| Bacteriophage X2 | AY954968 | ND | ND | ND |
| Bacteriophage bIL170 | AF009630 | putative major structural protein | 301 | NP_047126 |
| Bacteriophage bIL285 | AF323668 | capsid protein | 397 | NP_076616 |
| Bacteriophage bIL286 | AF323669 | capsid protein | 408 | NP_076679 |
| Bacteriophage bIL309 | AF323670 | capsid protein | 437 | NP_076738 |
| Bacteriophage bIL310 | AF323671 | ND | ND | ND |
| Bacteriophage bIL311 | AF323672 | ND | ND | ND |
| Bacteriophage bIL312 | AF323673 | ND | ND | ND |
| Bacteriophage c-st | AP008983 | ND | ND | ND |
| Bacteriophage phBC6A51 | NC_004820 | ND | ND | ND |
| Bacteriophage phBC6A52 | NC_004821 | ND | ND | ND |
| Bacteriophage phi AT3 | AY605066 | putative major head protein | 394 | YP_025031 |
| Bacteriophage phi CTX | AB008550 | predicted major capsid protein | 338 | NP_490602 |
| Bacteriophage phi ETA | AP001553 | similar to phage B1 major head | 274 | NP_510938 |
| | | protein | | |
| Bacteriophage phi JL001 | AY576273 | coat protein | 374 | YP_223991 |
| Bacteriophage phi LC3 | AF242738 | major head protein | 298 | NP_996706 |
| Bacteriophage phi-105 | AB016282 | ND | ND | ND |
| Bacteriophage phi-12 segment L | AF408636 | ND | ND | ND |
| Bacteriophage phi-12 segment M | AY039807 | ND | ND | ND |
| Bacteriophage phi-12 segment S | AY034425 | nucleocapsid protein P8 | 192 | NP_690826 |
| Bacteriophage phi-8 segment L | AF226851 | ND | ND | ND |
| Bacteriophage phi-8 segment M | AF226852 | ND | ND | ND |
| Bacteriophage phi-8 segment S | AF226853 | ND | ND | ND |
| Bacteriophage phi-BT1 | AJ550940 | ND | ND | ND |
| Bacteriophage phi-C31 | AJ006589 | ND | ND | ND |
| Bacteriophage phi1026b | AY453853 | ND | ND | ND |
| Bacteriophage phi3626 | AY082070 | major capsid protein | 421 | NP_612835 |
| Bacteriophage phiE125 | AF447491 | putative major capsid protein | 435 | NP_536362 |
| Bacteriophage phiKMV | AJ505558 | capsid protein | 335 | NP_877471 |
| Bacteriophage phiKO2 | AY374448 | major capsid head protein precursor | 428 | YP_006586 |
| Bacteriophage phiMFV1 | AY583236 | ND | ND | ND |
| Bacteriophage phiYeO3-12 | AJ251805 | major capsid protein 10A | 347 | NP_052109 |
| Bacteriophage phig1e | X98106 | minor capsid protein | 261 | NP_695154 |
| Bacteriophage r1t | U38906 | ND | ND | ND |
| Bacteriophage sk1 | AF011378 | ND | ND | ND |
| Bordetella phage BIP-1 | AY526909 | ND | ND | ND |
| Bordetella phage BMP-1 | AY526908 | ND | ND | ND |
| Bordetella phage BPP-1 | AY029185 | ND | ND | ND |
| Burkholderia cenocepacia phage Bcep1 | AY369265 | ND | ND | ND |
| Burkholderia cenocepacia phage BcepB1A | AY616033 | ND | ND | ND |
| Burkholderia cenocepacia phage BcepMu | AY539836 | ND | ND | ND |
| Burkholderia cepacia complex phage BcepC6B | AY605181 | ND | ND | ND |
| Burkholderia cepacia phage Bcep176 | DQ203855 | ND | ND | ND |
| Burkholderia cepacia phage Bcep22 | AY349011 | ND | ND | ND |
| Burkholderia cepacia phage Bcep43 | AY368235 | ND | ND | ND |
| Burkholderia cepacia phage Bcep781 | AF543311 | ND | ND | ND |
| Burkholderia cepacia phage BcepNazgul | AY357582 | putative capsid protein | 346 | NP_918991 |
| Burkholderia pseudomallei phage phi52237 | DQ087285 | phage major capsid protein | 337 | YP_293748 |
| Chlamydia phage 2 | AJ270057 | VP1 structural protein | 565 | NP_054647 |
| Chlamydia phage 3 | AJ550635 | Capsid protein (F protein)″ | 565 | YP_022479 |
| Chlamydia phage 4 | AY769964 | putative major coat protein | 554 | YP_338238 |
| Chlamydia phage PhiCPG1 | U41758 | capsid protein VP3 | 148 | NP_510875 |
| Chlamydia phage phiCPAR39 | AE002163 | capsid protein VP3 | 148 | NP_063897 |
| Chlamydia psittaci bacteriophage chp1 | D00624 | Capsid protein VP2 | 263 | NP_044314 |
| Coliphage ID11 | AY751298 | ND | ND | ND |
| Coliphage alpha3 | X60322 | major coat protein | 431 | NP_039597 |
| Coliphage phiK | X60323 | major coat protein | 431 | NP_043949 |
| Coliphage phiX174 | J02482 | F; major coat protein | 427 | NP_040711 |
| Cyanophage P-SSM2 | AY939844 | T4-like major capsid protein | 470 | YP_214367 |
| Cyanophage P-SSM4 | AY940168 | ND | ND | ND |
| Cyanophage P-SSP7 | AY939843 | T7-like capsid protein | 375 | YP_214206 |
| Cyanophage P60 | AF338467 | minor capsid protein | 221 | NP_570347 |
| Enterobacteria phage 186 | U32222 | major capsid protein | 355 | NP_052253 |
| Enterobacteria phage FI | X07489 | coat protein | 132 | NP_695027 |
| Enterobacteria phage G4 | J02454 | major coat protein | 427 | NP_040678 |
| Enterobacteria phage GA | D10027 | coat protein | 130 | NP_040754 |
| Enterobacteria phage HK022 | AF069308 | major capsid subunit precursor | 385 | NP_037666 |
| Enterobacteria phage I2-2 | X14336 | ND | ND | ND |
| Enterobacteria phage If1 | U02303 | major coat protein | 74 | NP_047355 |
| Enterobacteria phage Ike | X02139 | G VI capsid protein | 116 | NP_040577 |
| Enterobacteria phage K1E | AM084415 | ND | ND | ND |
| Enterobacteria phage K1F | DQ111067 | capsid | 347 | YP_338120 |
| Enterobacteria phage KU1 | AF227250 | coat protein | 130 | NP_057948 |
| Enterobacteria phage L17 | AY848684 | major capsid protein | 395 | YP_337933 |
| Enterobacteria phage M13 | V00604 | ND | ND | ND |
| Enterobacteria phage Mu | AF083977 | major head subunit | 305 | NP_050638 |
| Enterobacteria phage P1 | AF234172 | ND | ND | ND |
| Enterobacteria phage P2 | AF063097 | ND | ND | ND |
| Enterobacteria phage P22 | BK000583 | coat protein | 430 | NP_059630 |
| Enterobacteria phage PR3 | AY848685 | major capsid protein | 395 | YP_337964 |
| Enterobacteria phage PR4 | AY848686 | major capsid protein | 395 | YP_337995 |
| Enterobacteria phage PR5 | AY848687 | major capsid protein | 395 | YP_338026 |
| Enterobacteria phage PR772 | AY848688 | major capsid protein | 395 | YP_338057 |
| Enterobacteria phage PRD1 | M69077 | ND | ND | ND |
| Enterobacteria phage RB43 | AY967407 | gp23 precursor of major head | 524 | YP_239203 |
| | | subunit | | |
| Enterobacteria phage RB49 | AY343333 | major capsid protein | 528 | NP_891732 |
| Enterobacteria phage RB69 | AY303349 | gp23 major head protein | 522 | NP_861877 |
| Enterobacteria phage S13 | M14428 | capsid protein | 427 | NP_040750 |
| Enterobacteria phage SP6 | AY288927 | major capsid protein | 401 | NP_853592 |
| Enterobacteria phage Sf6 | AF547987 | ND | ND | ND |
| Enterobacteria phage T1 | AY216660 | putative major head subunit | 370 | YP_003895 |
| | | precursor | | |
| Enterobacteria phage T4 | AF158101 | gp23 major head protein | 521 | NP_049787 |
| Enterobacteria phage T7 | V01146 | major capsid protein | 345 | NP_041998 |
| Enterobacteria phage epsilon15 | AY150271 | ND | ND | ND |
| Enterobacteria phage fr | X15031 | coat protein | 130 | NP_039624 |
| Enterobacteria phage lambda | J02459 | capsid component | 533 | NP_040583 |
| Enterobacterio phage MS2 | J02467 | coat protein | 130 | NP_040648 |
| Enterobacteriophage Qbeta | AF059242 | major coat protein | 133 | NP_046751 |
| Haemophilus phage HP1 | U24159 | ND | ND | ND |
| Haemophilus phage HP2 | AY027935 | capsid | 336 | NP_536823 |
| Halovirus HF2 | AF222060 | ND | ND | ND |
| Lactobacillus bacteriophage phi adh | AJ131519 | major head protein | 395 | NP_050151 |
| Lactobacillus casei bacteriophage A2 | AJ251789 | major head protein | 400 | NP_680487 |
| Lactobacillus johnsonii prophage Lj928 | AY459533 | putative major head protein | 111 | NP_958536 |
| Lactobacillus johnsonii prophage Lj965 | AY459535 | putative major head protein | 349 | NP_958585 |
| Lactobacillus plantarum bacteriophage LP65 | AY682195v | ND | ND | ND |
| Lactobacillus plantarum bacteriophage phiJL-1 | AY236756 | major head protein | 286 | YP_223889 |
| Lactococcus lactis bacteriophage TP901-1 | AF304433 | ND | ND | ND |
| Lactococcus lactis bacteriophage ul36 | AF349457 | major capsid protein | 287 | NP_663677 |
| Lactococcus phage BK5-T | AF176025 | major structural protein | 404 | NP_116499 |
| Lactococcus phage P335 | AF489521 | major structural protein | 408 | NP_839926 |
| Lactococcus phage c2 | L48605 | major capsid (head) protein | 480 | NP_043553 |
| Listeria bacteriophage P100 | DQ004855 | ND | ND | ND |
| Listeria phage 2389 (Bacteriophage PSA) | AJ312240 | major capsid protein a | 390 | NP_510986 |
| Listonella pelagia phage phiHSIC | AY772740 | major capsid protein | 315 | YP_224246 |
| Methanobacterium phage psiM2 | AF065411 | ND | ND | ND |
| Methanothermobacter wolfeii prophage psiM100 | AF301375 | ND | ND | ND |
| Mycobacteriophage Barnyard | AY129339 | ND | ND | ND |
| Mycobacteriophage Bxb1 | AF271693 | ND | ND | ND |
| Mycobacteriophage Bxz1 | AY129337 | ND | ND | ND |
| Mycobacteriophage Bxz2 | AY129332 | ND | ND | ND |
| Mycobacteriophage CJW1 | AY129331 | ND | ND | ND |
| Mycobacteriophage Che8 | AY129330 | ND | ND | ND |
| Mycobacteriophage Che9c | AY129333 | ND | ND | ND |
| Mycobacteriophage Che9d | AY129336 | ND | ND | ND |
| Mycobacteriophage Corndog | AY129335 | ND | ND | ND |
| Mycobacterium D29 | AF022214 | major head subunit; gp17 | 318 | NP_046832 |
| Mycobacteriophage Omega | AY129338 | ND | ND | ND |
| Mycobacteriophage PG1 | AF547430 | ND | ND | ND |
| Mycobacteriophage Rosebush | AY129334 | ND | ND | ND |
| Mycobacteriophage TM4 | AF068845 | major capsid subunit gp9 | 305 | NP_569745 |
| Mycobacterium phage L5 | Z18946 | ND | ND | ND |
| Mycoplasma arthritidis bacteriophage MAV1 | AF074945 | ND | ND | ND |
| Mycoplasma virus P1 | AF246223 | ND | ND | ND |
| Phage phi 4795 | AJ487680 | ND | ND | ND |
| Phage phiMHZK | AF306496 | major viral coat protein | 533 | NP_073538 |
| Phage phiSMA9 | AM040673 | ND | ND | ND |
| Propionibacterium phage phiB5 | AF428260 | Putative coat protein | 57 | NP_604425 |
| Pseudomonas aeruginosa bacteriophage PaP2 | AY575774 | ND | ND | ND |
| Pseudomonas aeruginosa phage F116 | AY625898 | ND | ND | ND |
| Pseudomonas aeruginosa phage PaP3 | AY078382 | major head protein | 317 | NP_775251 |
| Pseudomonas bacteriophage phi-13 segment L | AF261668 | P1 procapsid protein | 801 | NP_690819 |
| Pseudomonas bacteriophage phi-13 segment M | AF261667 | ND | ND | ND |
| Pseudomonas bacteriophage phi-13 segment S | AF261666 | P8 nucleocapsid shell protein | 151 | NP_690807 |
| Pseudomonas phage D3 | AF165214 | major head protein | 395 | NP_061502 |
| Pseudomonas phage PP7 | X80191 | coat protein | 128 | NP_042305 |
| Pseudomonas phage Pf1 | X52107 | major coat protein | 82 | NP_039603 |
| Pseudomonas phage Pf3 | M11912 | major coat protein | 44 | NP_040652 |
| Pseudomonas phage gh-1 | AF493143 | major capsid protein A | 347 | NP_813774 |
| Pseudomonas phage phi-6 segment L | M17461 | ND | ND | ND |
| Pseudomonas phage phi-6 segment M | M17462 | ND | ND | ND |
| Pseudomonas phage phi-6 segment S | M12921 | ND | ND | ND |
| Pseudomonas phage phiEL | AJ697969 | ND | ND | ND |
| Pseudomonas phage phiKZ | AF399011 | ND | ND | ND |
| Ralstonia phage p12J | AY374414 | ND | ND | ND |
| Roseophage SIO1 | AF189021 | ND | ND | ND |
| SVTS2 plectrovirus | AF133242 | ND | ND | ND |
| Salmonella typhimurium bacteriophage ES18 | AY736146 | ND | ND | ND |
| Salmonella typhimurium bacteriophage ST104 | AB102868 | ND | ND | ND |
| Salmonella typhimurium bacteriophage ST64T | AY052766 | ND | ND | ND |
| Salmonella typhimurium phage ST64B | AY055382 | Major capsid protein precursor | 401 | NP_700379 |
| Shigella flexneri bacteriophage V | U82619 | capsid | 409 | NP_599037 |
| Sinorhizobium meliloti phage PBC5 | AF448724 | ND | ND | ND |
| Spiroplasma phage 1-C74 | U28974 | ND | ND | ND |
| Spiroplasma phage 1-R8A2B | X51344 | ND | ND | ND |
| Spiroplasma phage 4 | M17988 | ND | ND | ND |
| Staphylococcus aureus bacteriophage PVL | AB009866 | capsid protein | 415 | NP_058445 |
| Staphylococcus aureus phage phi 11 | AF424781 | head protein | 324 | NP_803287 |
| Staphylococcus aureus phage phi 12 | AF424782 | ND | ND | ND |
| Staphylococcus aureus phage phi 13 | AF424783 | head protein | 415 | NP_803388 |
| Staphylococcus aureus phage phiP68 | AF513033 | major head protein | 408 | NP_817336 |
| Staphylococcus aureus prophage phiPV83 | AB044554 | ND | ND | ND |
| Staphylococcus aureus temperate phage phiSLT | AB045978 | ND | ND | ND |
| Staphylococcus phage 44AHJD | AF513032 | major head protein | 408 | NP_817314 |
| Staphylococcus phage K | AY176327 | putative capsid protein | 463 | YP_024474 |
| Staphylococcus phage Twort | AY954970 | ND | ND | ND |
| Staphylococcus phage phiN315 | NC004740 | ND | ND | ND |
| Streptococcus mitis phage SM1 | AY007505 | ND | ND | ND |
| Streptococcus phage C1 | AY212251 | major capsid protein | 392 | NP_852022 |
| Streptococcus phage Cp-1 | Z47794 | major head protein | 365 | NP_044821 |
| Streptococcus pneumoniae bacteriophage MM1 | AJ302074 | putative minor capsid protein 1 | 522 | NP_150162 |
| Streptococcus pyogenes phage 315.1 | NC_004584 | major coat protein | 377 | NP_795405 |
| Streptococcus pyogenes phage 315.2 | NC_004585 | ND | ND | ND |
| Streptococcus pyogenes phage 315.3 | NC_004586 | ND | ND | ND |
| Streptococcus pyogenes phage 315.4 | NC_004587 | putative major capsid/head protein | 272 | NP_795582 |
| Streptococcus pyogenes phage 315.5 | NC_004588 | ND | ND | ND |
| Streptococcus pyogenes phage 315.6 | NC_004589 | ND | ND | ND |
| Streptococcus thermophilus bacteriophage 2972 | AY699705 | head protein | 297 | YP_238489 |
| Streptococcus thermophilus bacteriophage 7201 | AF145054 | ND | ND | ND |
| Streptococcus thermophilus bacteriophage DT1 | AF085222 | major head protein | 293 | NP_049396 |
| Streptococcus thermophilus bacteriophage Sfi11 | AF158600 | ND | ND | ND |
| Streptococcus thermophilus bacteriophage Sfi19 | AF115102 | major head protein | 397 | NP_049929 |
| Streptococcus thermophilus bacteriophage Sfi21 | AF115103 | major head protein | 397 | NP_049971 |
| Streptococcus thermophilus temperate | U88974 | ND | ND | ND |
| bacteriophage O1205 | | | | |
| Stx1 converting bacteriophage virion | AP005153 | ND | ND | ND |
| Stx2 converting bacteriophage I | AP004402 | ND | ND | ND |
| Stx2 converting bacteriophage II | AP005154 | ND | ND | ND |
| Sulfolobus islandicus filamentous | AF440571 | putative outer membrane protein | 212 | NP_445721 |
| Sulfolobus islandicus rod-shaped virus 1 | AJ414696 | ND | ND | ND |
| Sulfolobus islandicus rod-shaped virus 2 | AJ344259 | ND | ND | ND |
| Sulfolobus spindle-shaped virus 1 | X07234 | ND | ND | ND |
| Sulfolobus spindle-shaped virus 2 | AY370762 | ND | ND | ND |
| Sulfolobus spindle-shaped virus Kamchatka-1 | AY423772 | ND | ND | ND |
| Sulfolobus spindle-shaped virus Ragged Hills | AY388628 | ND | ND | ND |
| Sulfolobus tengchongensis spindle-shaped virus | AJ783769 | ND | ND | ND |
| STSV1 | | | | |
| Sulfolobus turreted icosahedral virus | AY569307 | ND | ND | ND |
| Temperate phage PhiNIH1.1 | AY050245 | major capsid protein | 272 | NP_438146 |
| Vibrio cholerae O139 fs1 phage | D89074 | ND | ND | ND |
| Vibrio cholerae filamentous bacteriophage fs-2 | AB002632 | putative capsid protein | 116 | NP_047370 |
| Vibrio cholerae phage KSF-1phi | AY714348 | ND | ND | ND |
| Vibrio cholerae phage VGJphi | AY242528 | putative major capsid protein | 44 | NP_835475 |
| Vibrio harveyi bacteriophage VHML | AY133112 | ND | ND | ND |
| Vibrio phage VSK | AF453500 | major coat protein | 49 | NP_752644 |
| Vibrio phage Vf12 | AB012574 | ND | ND | ND |
| Vibriophage VP2 | AY505112 | outer capsid protein | 460 | YP_024425 |
| Vibriophage VP4 | DQ029335 | Major capsid protein | 324 | YP_249589 |
| Vibriophage VP5 | AY510084 | ND | ND | ND |
| Vibriophage VpV262 | AY095314 | ND | ND | ND |
| Virus PhiCh1 | AF440695 | capsid protein | 467 | NP_665924 |
| Xanthomonas campestris pv. pelargonii phage Xp15 | AY986977 | ND | ND | ND |
| Xanthomonas oryzae bacteriophage Xp10 | AY299121 | head protein; major capsid subunit | 390 | NP_858956 |
| | | precursor | | |
| Xanthomonas oryzae phage OP1 | AP008979 | putative head protein | 390 | YP_453565 |
| Xanthomonas oryzae phage OP2 | AP008986 | putative head protein | 303 | YP_453628 |
| Xanthomonas phage Cflc | M57538 | A coat protein | 419 | NP_536675 |
| Yersinia pestis phiA1122 | AY247822 | major capsid protein | 344 | NP_848297 |

*ND = Not determined

### Biotin-Specific Ligands

The interaction of egg white avidin and bacterial streptavidin with biotin has evolved into an indispensable tool for general use in the biological sciences and as a model for the study of the interaction of a ligand with a protein. Both avidin and streptavidin bind biotin with an essentially immeasurably high affinity constant. The affinity constant for avidin has been estimated at approximately 10¹⁵M⁻¹ and that for streptavidin at 1-2 orders of magnitude lower.

The highly specific interaction of avidin with the small vitamin biotin can be a useful tool in assay systems designed to detect and target biological analytes. The extraordinary affinity of avidin for biotin allows biotin-containing molecules in a complex mixture to be discretely bound with avidin conjugates.

Chickens are known to produce several different proteins which bind biotin in a non-covalent fashion. One of them is avidin, which is expressed by oviduct cells upon progesterone induction and is then transferred to the egg-white where it constitutes a minor fraction of the total protein content of the egg-white. Another biotin-binder, called literally biotin-binding protein (BBP), is presumably induced by estrogen and secreted from the liver into chicken plasma. From plasma, the BBP is thought to be deposited in egg-yolk. Another egg-white BBP, distinct from avidin, has biochemical characteristics that resemble those reported for yolk BBP (Seshagiri, P. B. and Adiga, P. R. 1987 Biochim Biophys Acta 926:321-330).

Avidin is a glycoprotein found in the egg white and tissues of birds, reptiles and amphibians. This protein contains four identical subunits having a combined mass of 67,000-68,000 daltons. Each subunit consists of 128 amino acids and binds one molecule of biotin. Avidin is highly glycosylated: carbohydrate accounts for about 10% of the total mass of avidin. Avidin has a basic isoelectric point (pI) of 10-10.5 and is very soluble in water and aqueous salt solutions. Avidin is stable over a wide range of pH and temperature. Extensive chemical modification has little effect on the activity of avidin, making it useful for detection and protein purification.

Streptavidin is another biotin-binding protein that is isolated from Streptomyces avidinii and has a mass of 60,000 daltons. In contrast to avidin, streptavidin has no carbohydrate and has an acidic isoelectric point (pI=5). Streptavidin is much less soluble in water than avidin and can be crystallized from water or 50% isopropyl alcohol. There are considerable differences in the composition of avidin and streptavidin, but they are remarkably similar in other respects. Streptavidin is also a tetrameric protein, with each subunit binding one molecule of biotin with a similar affinity to that of avidin. Guanidinium chloride will dissociate avidin and streptavidin into subunits, but streptavidin is more resistant to dissociation.

### Bioconjugates

Bioconjugate is a generic term to describe detection reagents coupled to proteins, oligonucleotides, small molecules, etc. that are used to direct binding of the detection reagent to an area of interest. Detection reagents (e.g., stains, enzymes and fluorescent nanocrystals) that may be used include, but are not limited to, the fluorescent probe ALEXA (available from Molecular Probes, Inc., Eugene, Oreg.), Cy3, fluorescein isothiocyanate, tetramethylrhodamine, horseradish peroxidase, alkaline phosphatase, glucose oxidase, fluorescent semiconductor nanocrystals (e.g., quantum dots (QDs)) or any other label known in the art. Some proteins for bioconjugation encompass streptavidin, avidin, or protein A.

QD bioconjugate is a generic term used to describe QD nanocrystals coupled to proteins, oligonucleotides, small molecules, etc. which are used to direct binding of the quantum dots to areas of interest. “Qdot®” is a registered trademark belonging to Invitrogen (Quantum Dot Corporation, Hayward, Calif., U.S.A.). Examples of QD bioconjugates include streptavidin, protein A, and biotin families of QD conjugates. QD bioconjugates are often used as simple replacements for analogous conventional dye conjugates when superior performance is required to achieve lower limits of detection, more quantitative results, more photo-stable samples, higher levels of multiplexability, or any of the other advantages afforded by quantum dot technology.

Standard fluorescence microscopes are a useful tool for the detection of QD bioconjugates. These microscopes are often fitted with bright white light lamps and filter arrangements. QD nanocrystals are efficient at absorbing white light using broad excitation filters. Since QD conjugates are virtually completely photo-stable, time can be taken with the microscope to find regions of interest and to adequately focus on the samples. QD conjugates are useful any time bright photo-stable emission is required and are particularly useful in multicolor applications where only one excitation source/filter is available and minimal crosstalk among the colors is required.

### Functionalization of Bioconjugates

To create protein bioconjugates for various assays, a variety of proteins have been either covalently attached or electrostatically self-assembled onto fluorescent semiconductor nanocrystal surfaces (Behrens, S. et al. 2002 Adv Mater 14:1621-1625; Mao, C. B. et al. 2003 Proc Natl Acad Sci USA 100:6946-6951; Chan, W. C. W. & Nie, S. M. 1998 Science 281:2016-2018; Goldman, E. R. et al. 2002 J Am Chem Soc 124:6378-6382; Goldman, E. R. et al. 2002 Anal Chem 74:841-847; Ishii, D. et al. 2003 Nature 423:628-632; Mattoussi, H. et al. 2000 J Am Chem Soc 122:12142-12150; Akerman, M. E. et al. 2002 Proc Natl Acad Sci USA 99:12617-12621; Kloepfer, J. A. et al. 2003 Appl Environ Microbiol 69:4205-4213; Wang, L. Y. et al. 2002 Analyst 127:1531-1534; Lin, Z. B. et al. 2003 Anal Biochem 319:239-243; and Dahan, M. et al. 2003 Science 302:442-445)

### Attachment Sites

In some embodiments of the invention, bacteriophage are engineered to have the major head, capsid protein assembly of the phage express a first attachment site. Additionally a bioconjugate is functionalized with a second attachment site capable of recognizing/binding the first attachment site expressed on the engineered phage. The first attachment site may be a protein, a polypeptide, a sugar, a polynucleotide, a natural or synthetic polymer, a secondary metabolite or compound (biotin, fluorescein, retinol, digoxigenin, metal ions, phenylmethysulfonylfluoride), or a combination thereof, or a chemically reactive group thereof. The second attachment site of the bioconjugate or reagent that is to be linked to the bacteriophage may be a protein, a polypeptide, a sugar, a polynucleotide, a natural or synthetic polymer, a secondary metabolite or compound (biotin, fluorescein, retinol, digoxigenin, metal ions, phenylmethysulfonylfluoride), or a combination thereof, or a chemically reactive group thereof.

First and second attachment sites include, but are not limited to, binding pairs such as biotin/streptavidin, antigen/antibody, receptor/ligand partners, protein A/antibody Fc domain, and leucine zipper domains (e.g., JUN-FOS leucine zipper domain).

### Assay Conditions

In one embodiment, a first step is to add non-biotinylated bacteriophage to the test sample. The target bacterial cells are infected when they come into contact with the phage. Infected bacterial cells are incubated under conditions to form biotinylated bacteriophage. Biotinylated bacteriophage may be detected by a variety of means. For example, biotinylated bacteriophage may be detected by contacting the solution with a biotin-specific ligand conjugated bioconjugate. Biotinylated bacteriophage may be concentrated prior to contacting with a biotin-specific ligand conjugated bioconjugate. The presence of biotinylated bacteriophage in the sample indicates the presence of target bacterial cells in the sample and the absence of biotinylated bacteriophage indicates the absence of target bacterial cells in the sample.

In another embodiment, a first step is to add a complex to the test sample in which the complex combines a biotinylated bacteriophage and conjugation of the phage to a biotin-specific ligand conjugated bioconjugate. The target bacterial cells are bound when they come into contact with the complex. The complex may be detected by a variety of means. The presence of complex-bound bacteria in the sample indicates the presence of target bacterial cells in the sample and the absence of complex-bound bacteria indicates the absence of target bacterial cells in the sample.

Preferably throughout detection assays, the sample is maintained at a temperature that maintains the viability of any pathogen cell present in the sample. During steps in which bacteriophage are attaching to bacterial cells, it is preferable to maintain the sample at a temperature that facilitates bacteriophage attachment. During steps in which bacteriophage are replicating within an infected bacterial cell or lysing such an infected cell, it is preferable to maintain the sample at a temperature that promotes bacteriophage replication and lysis of the host. Such temperatures are at least about 25° C., more preferably no greater than about 45° C., most preferably about 37° C. It is also preferred that the samples be subjected to gentle mixing or shaking during bacteriophage attachment, replication and release.

Assays may include various appropriate control samples. For example, control samples containing no bacteriophage or control samples containing bacteriophage without bacteria may be assayed as controls for background levels.

### High Sensitivity Bacterial Detection Using Biotin Tagged Phage and Quantum-Dot Nanocomplex

### Overall Strategy and Considerations for the Detection Method

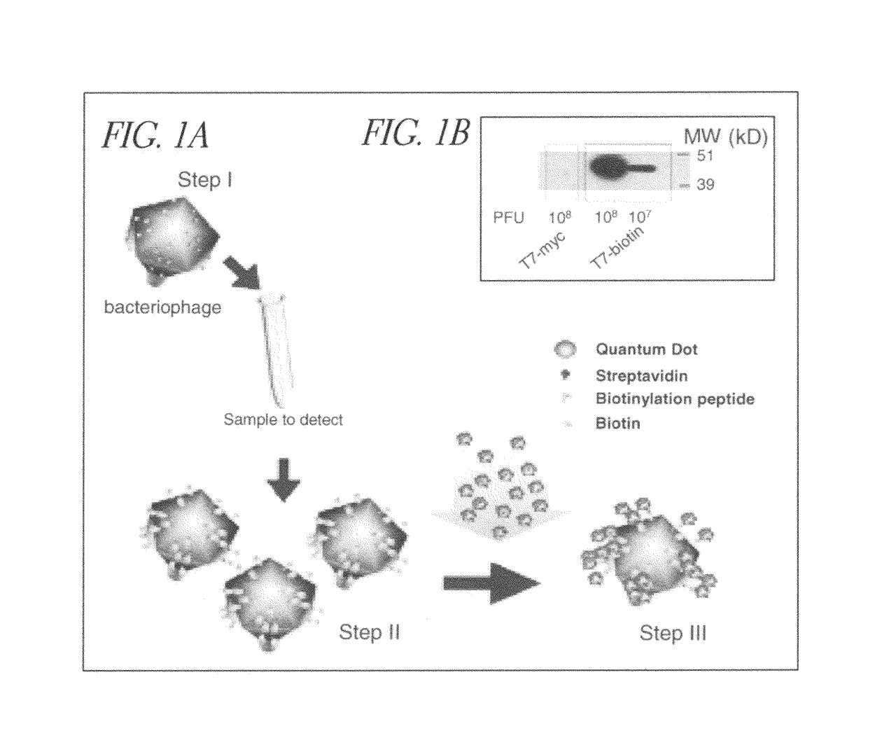

Our strategy of the detection method is shown in FIG. 1a. We engineered a phage to display a small peptide, that can be biotinylated (biotinylation peptide), fused to the major capsid protein. Our “reagent” phage (step I) contains the genetic information to display tagged head protein but is assembled either (i) in vivo in a nonbiotinylating mutant host to display nonbiotinylated biotinylation peptide or (ii) in vitro, to contain the wild-type capsid protein (see below). If the specific bacteria sensitive to these phage are present in the sample, upon addition of the phage, the latter will infect the bacteria and produce progeny phage during which time the cell's biotin-ligase protein, BLP (BirA in case of E. coli used in our experiments), will recognize the biotinylation peptide and ligate biotin to it. Biotin (vitamin H), which is present in all living cells, is attached post-translationally by BLP to a specific lysine residue in the tagged peptide (Kwon, K., & Beckett, D. 2000 Protein Sci 9:1530-1539). The biotinylation of the target protein(s) by BLP is extremely conserved throughout evolution (Kwon, K., & Beckett, D. 2000 Protein Sci 9:1530-1539; Chapman-Smith, A. & Cronan Jr., J.-E. 1999 Biomol Eng 16:119-125). The use of such a highly conserved pathway will enable biotinylation of any such “reagent” phage in its corresponding bacterial species. Phage will assemble and incorporate the biotinylated capsid-peptide fusion protein to their head, followed by lysis and release of phage particles displaying the biotinylated peptide (step II). Newly released phage are readily distinguishable from the leftover unabsorbed “reagent” phage in the sample by their biotinylation. For every bacterium in the sample, a high degree of amplification will occur depending on the burst size of the phage. In step III, the presence of biotinylated phage particles in the lysate, which reflect the presence of sensitive bacteria in the original sample, is detected by conjugation to streptavidin-functionalized QDs.

### Engineering the “Reagent” Phage

As a model system, we engineered the coliphage T7 to express the major capsid protein, gp10A, fused to the 15-amino acid biotinylation peptide (T7-bio): GLNDIFEAQKIEWHE (SEQ ID NO: 4) (Cull, M.-G. & Schatz, P.-J. 2000 Methods Enzyniol 326:430-440). This cloning strategy (See Example 1 for details) results in the display of the given peptide on all 415 monomers of the major capsid protein. To differentiate the “reagent” phage from phage released from infected target cells, it is crucial that the reagent input phage is not biotinylated. We accomplished this in one of the two ways: (i) by packaging the engineered T7 phage DNA in vitro using wild-type virion proteins or (ii) by propagating our reporter phage on a biotin auxotroph. In the first method we used a commercially available packaging kit composed of wild type phage proteins (T7Select, Novagen). This system when used with the engineered phage T7 DNA gave rise to 10³-10⁶ plaque forming units (PFU)/1 μg DNA. In the second method, we prepared phage lysates by two rounds of growth on an E. coli biotin auxotroph that was starved for biotin as was evident by an inhibition of bacterial growth that addition of biotin could relieve. The absence of biotinylated phage resulting from either method of production was confirmed by western blot analysis (using streptavidin-HRP) and fluorescence microscope using streptavidin-QDs (Table 3, last row).

TABLE 3
Detecting E. coli among several different bacterial cells

| | | Expected number of | Relative number of QD |
|---|---|---|---|
| Number of | Number of | progeny phage/ | bound E. coli detected/ |
| E. coli cells | other cells | ml⁽*¹⁾ | ml⁽*²⁾, % |
| 10⁷ | 0 | 10⁹ | 100 |
| 10⁵ | 10⁷ | 10⁷ | 66 |
| 10³ | 10⁷ | 10⁵ | 49 |
| 10² | 10⁷ | 10⁴ | 31 |
| 10 | 10⁷ | 10³ | 28 |
| 0 | 10⁷ | 0 | 8 |
| 10⁷⁽*³⁾ | 0 | 0 | 6 |
| 0⁽*⁴⁾ | 0 | 0 | 1 |

⁽*¹⁾Based on burst size of 100 phage/cell.
⁽*²⁾Only E. coli normalized to 100.
⁽*³⁾No phage added.
⁽*⁴⁾Non-biotinylated phage added at the detection step.

The resulting nonbiotinylated “reagent” phage, when making progeny particles following infection of wild type E. coli bacterial cells, gets biotinylated at the displayed peptide by the host BLP resulting in biotinylated phage referred to as T7-bio. As a negative control, we engineered T7 phage to express the major capsid protein fused to the 10-amino acid myc peptide (T7-myc), EQKLISEEDL (SEQ ID NO: 5), which results in a phage that displays the myc peptide but is not recognized by the host BLP.

### The Phage Displayed Peptide is Biotinylated by the Phage Specific Host In Vivo and can Bind Quantum Dots

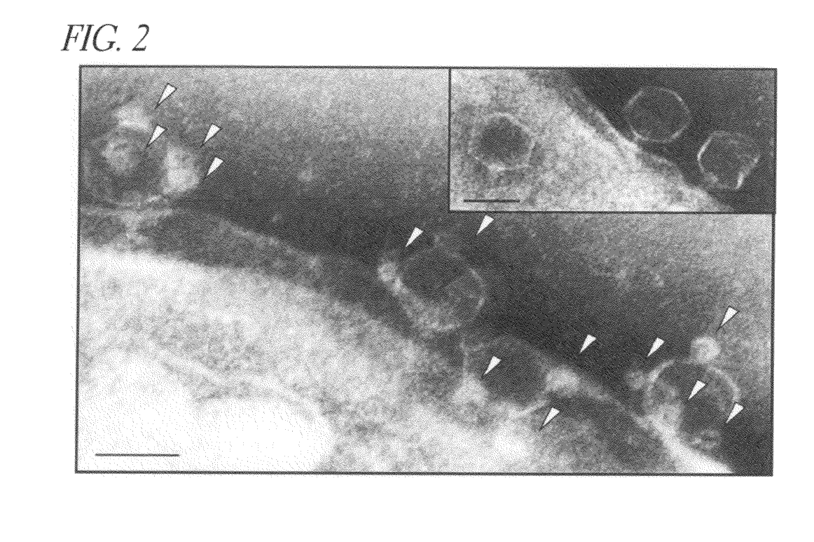

We tested the ability of the engineered phage to infect E. coli and become biotinylated, by mixing the phage with a bacterial culture until cell lysis was visible. The presence of the biotin molecules on the phage's displayed peptide was initially detected by western blot analysis of the virion proteins from purified phage samples using streptavidin-HRP. The western blot confirmed the in vivo biotinylation of the tagged capsid protein assembled on the phage head (FIG. 1b). We used transmission electron microscope (TEM) to obtain a quantitative estimation for the number of biotinylated peptides on each phage. We adsorbed phage to carrier bacterial cells, conjugated streptavidin-coated QDs (in excess) to the phage, and removed free QDs by centrifugation and washing steps. Binding of QDs (arrowheads) to phage T7-bio was clearly demonstrable (FIG. 2) while control phage, T7-myc, did not show bound QDs (FIG. 2 inset). We estimated the average number of QDs/phage to be 2.2 (±1.3) (0.5% of the total peptides displayed), while an estimated 7% of the T7-bio phage had no QDs bound. Recombinant proteins carrying a biotinylation peptide expressed in low and high amounts are typically biotinylated at about 30% and 6% efficiency, respectively, by endogenous levels of the BirA enzyme in E. coli (Cull, M.-G. & Schatz, P.-J. 2000 Methods Enzymol 326:430-440). The low level of biotinylation obtained in our case, 0.5%, can be attributed to the very short (13 min) latent time of phage T7 and/or, more likely, to the very high expression level of the capsid protein, which may be overwhelming BirA. In agreement with either explanation, when cells expressing BirA from a multicopy plasmid were used, a much higher level of bound biotin molecules/phage was obtained, as detected by western blot analysis. It might be useful to include the birA gene in the engineered phage to allow more of the displayed peptides to be biotinylated, thereby, to increase the detection sensitivity. Biotin is a small molecule that does not seem to interfere with phage head assembly and stability (see below). Nevertheless, QD are about 1/10 the size of the phage head, which indicates that only a limited number of QDs (up to about 100 QDs with maximum surface coverage) can fit on a single phage head surface. Importantly, neither inactivation nor aggregation of the phage bound to QDs was observed as tested by comparing the ability of phage to form plaques ±QDs; there was no decrease in PFU.

### Detection of QDs-Phage Complexes by Flow Cytometry

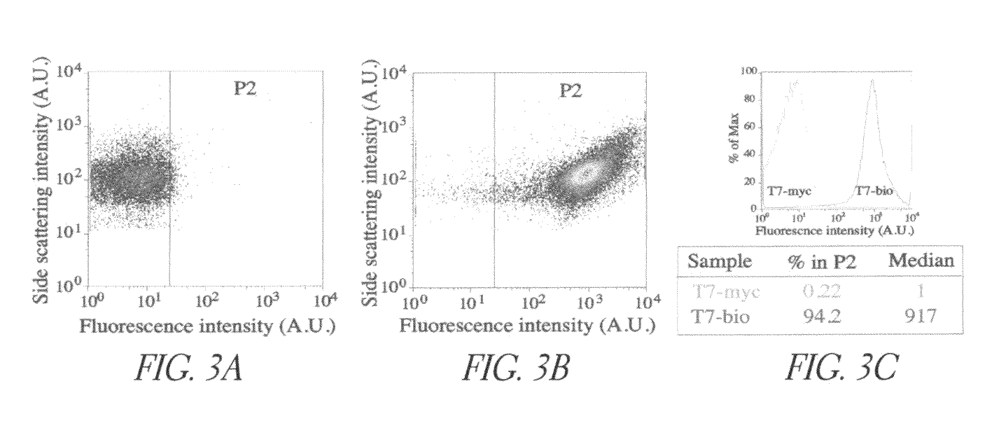

Biotinylated phage bound to QDs were initially detected and quantitatively analyzed by flow cytometry. Flow cytometry allow scanning of a large number of particles; single cells flow in a fast stream through a focal volume of an excitation laser beam and light intensities due to side scattering (SSC, measured at 90 degrees relative to the direction of the focused laser light), forward scattering (FSC, at 180 degrees), and fluorescence light (FL, at 90 degrees) are monitored. Since phage-QD complexes are too small to be detected by the SSC, we used carrier-cells to which the phage T7 can bind. In FIG. 3 scatter plots of FL vs. SSC from each of the 30,000 cells infected either by T7-myc+QD (3a) or by T7-bio+QDs (3b), at multiplicity of infection (MOI) of 5, are compared. The results show that T7-bio infected cells exhibit 2 orders of magnitude higher fluorescence than control, as a result of the binding of streptavidin-QDs to the biotins in the capsid of the T7-bio. Differentiation in fluorescence signal between the two populations is clear from the histograms of cells vs. fluorescence shown in FIG. 3c. Setting a threshold of m-2σ (22 A.U. in the FL channel) calculated from the histogram of the T7-myc infected cells (designated P2), about 94% of the T7-bio bound cells showed fluorescence intensities above the threshold while less then 1 percent of the T7-myc bound cells did so. These results confirm our TEM observations in a larger population, validating no binding of QDs to the control phage, T7-myc, and preferential conjugation of streptavidin-QDs to T7-bio. In addition, free QDs and/or phage-QD complexes that might be present in the sample are not detected since they do not trigger the detector channels. We estimate that the median in the flow cytometry measurement corresponds to 4 QDs: 2 QDs/phage (as estimated from TEM images) at MOI of 2. We believe that including the birA gene in the engineered phage, proposed above, would enhance the signal such that one phage/cell will be detected using the flow cytometry.

### Single QDs-Phage Complexes are Readily Detected by Fluorescence Microscopy: Quantized Blinking State Allows for the Visualization of a Single Bound Phage

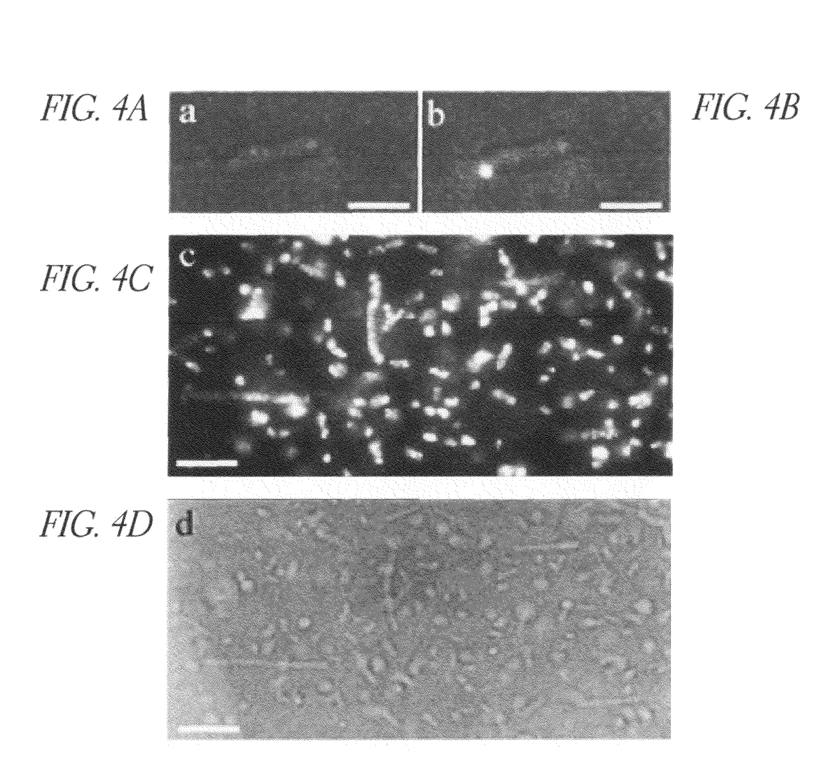

As a second method to detect QD labeled phage, we used fluorescence microscopy, which permits quantitative measurements with the sensitivity to detect a single QD conjugated to a single phage. As with the flow cytometry, we used carrier bacterial cells to allow removal of free QDs by washing. FIG. 4 shows optical micrographs of phage-QDs complexes bound to cells. FIGS. 4a and b are typical images of a single cell decorated with a single phage-QD complex, obtained as a result of using a low number of biotinylated phage in the sample. Imaging at two different quantized blinking states in which the single QD is in either “on” or “off” verifies that a single QD is present. FIGS. 4c and 4d demonstrate that the number of phage-QD complexes on every cell is higher when a high number of biotinylated phages are added. Quantitative measurements of MOI and the number of QDs on each phage may be possible, providing that the dispersion of phage-QD complexes is larger than the diffraction limit of the optical microscope, and as the optical characteristics of multiple QDs on a single phage-QD complex are the result of collective optical properties of single QDs. For instance, the number of quantized blinking steps in the fluorescence emission of a phage-QD complex will be directly correlated with the number of QDs in a single phage-QD complex (Yao, J. et al. 2005 Proc Natl Acad Sci USA 102:14284-14289). It is also noteworthy that no background fluorescence was observed from the cells or the medium (LB), and that the fluorescence emissions from QDs continued for hours without substantial photo-bleaching. When phages were omitted or when T7-myc was used under the same conditions, no fluorescence signal was observed.

Detection of E. Coli in a Mix with Other Bacteria and in Environmental Samples

To detect a small number of a given type of bacteria among several different bacterial cells, we used a culture with mixed bacterial strains on which phage T7 cannot propagate: Pseudomonas aeruginosa, Vibrio cholera, Salmonella, Yersinia pseudotuberculosis, and Bacillus subtilis. We analyzed mixtures of 2×10⁶ cells of each of the above strains mixed with different numbers of cells of Escherichia coli (from 10 to 10⁷ cells/ml). We followed the method as illustrated in FIG. 1 and scored the phage-QD conjugates by fluorescence microscopy. The results of such experiments, shown in Table 3, demonstrate that the non-E. coli strains provide a signal that is not significantly different from the background. The number of phage detected from a sample of only E. coli cells was normalized to 100% (all carrier cells contained QD). The number of phage detected was dependent upon the number of E. coli cells in the sample. When 1000 or 100 cells were detected, 52 out of 107 (49%) or 48 out of 155 (31%) carrier cells had conjugated T7-bio-QD, respectively. The signal from as few as 10 E. coli cells was significantly higher than the signal in controls with no E. coli or no phage added to the mix (124 out of 450 (28%), 1 out of 140 (<1%) and 45 out of 736 (6%) respectively).

Finally, we tested water samples from the Potomac River. We determined, in about one hour, that there are at least 20 E. coli cells in 1 ml of the sample. In comparison, using Coliscan MF kit (Micrology Laboratories, LLC, approved by the US Environmental Protection Agency), it took 24 hours to detect and identify 200 general coliforms in 1 ml of the same samples. The lower number of E. coli cells detected by our method is due to higher specificity of the phage to detect a particular coliform. These results demonstrate the rapid and specific nature of our assay.

### CONCLUSIONS

The primary significance of the current work is the development of a simple and highly sensitive procedure for phage-based bacterial detection that achieves (i) enhanced detection limit; (ii) rapidity; and (iii) broad applicability. Sensitivity is shown by our method's ability to detect and quantify low abundance targets of at least as few as 10 cells/ml. Our method takes about an hour to get results. The procedure uses biotinylation, a highly conserved pathway in nature, which can be applied to target a variety of bacteria in biological samples. Although we used a single phage-host system, the method may be expanded for the detection of multiple bacterial strains by their specific phages, each conjugated to QDs of different emission colors, in the same sample. Furthermore, higher specificity can be achieved by using multiple phages for one host, in the same sample, conjugated to QDs of different emission colors. The tools for detection can include microscopy, spectroscopy, or flow cytometry. It should be possible to utilize QD phage-based bacterial detection with hand-held instruments. Additionally, since phage could not be seen by light microscopy previously, and QD-labeled phage are infective, we believe that our method opens up new avenues to address phage biology-related questions on topics such as initial binding, phage localization, distribution and more.

### Example 1

### Engineering the T7-Bio and T7-Myc Phages

We used the T7Select System (Novagen) for engineering and packaging of DNA into T7 phage particles. For the T7-bio we used two phosphorylated primers: 3′L6bio and 5′L6bio, containing overhang sequences for ligation with HindIII and EcoRI digested phage arms DNA, respectively (upper case), a 6 amino acid linker coding sequence (underlined), followed by the biotinylation peptide coding DNA (lower case) and a stop codon (bold):

3′L6bio:
(SEQ ID NO: 6)
5′-AGCTTttagtgccattcgattttctgagcttcgaagatgtcgttca
ggcctgaaccacgcggccgcaacG-3′
5′L6bio:
(SEQ ID NO: 7)
5′-AATTCgttgcggccgcgtggttcaggcctgaacgacatcttcgaagc
tcagaaaatcgaatggcactaaA-3′.

The primers were annealed to each other by heating at 95° C. for 5 min in ligation buffer and cooling at room temperature, ligation to T7 arms was done as recommended by the manufacturer. For the engineering of the T7-myc phage we used the primers MYC1: 5′-AATTCtggtggcagcggatctgagcagaagctgatcagcgaggaagatcttaattaaA-3′ (SEQ ID NO: 8) and MYC2: 5′-AGCTttaattaagatcttcctcgctgatcagatctgctcagatccgctaccaccaG-3′ (SEQ ID NO: 9) containing overhang sequences for ligation with EcoRI and HindIII digested phage arms DNA (upper case), a 5 amino acid linker coding sequence (underlined), followed by the myc domain (lower case) and a stop codon (bold).

### Negative Stain for Transmission Electron Microscopy

Staining of phage was done as described elsewhere (Palmer, E.-L., & Martin, M.-L. 1988 CRC Press, Inc. Boca Raton, Fla. 154). Briefly, phage were incubated with E. coli for 4 min at 37° C. in PBS buffer. A streptavidin coated QD (QD 605) suspension, 1 μM, (Quantum Dot Corporation, Hayward, Calif., U.S.A.) was diluted 100 fold in PBS. 1 μl of the diluted solution was added and incubation continued for 5 min at room temperature. After centrifugation at 1500 rpm for 5 min, 1 μl of the sample was placed onto a carbon-coated Formvar-filmed copper grid (Tousimis Research Corp. Rockville, Md.) and allowed to attach. The sample was negatively stained with 1%, pH 7.0 phosphotungstic acid solution (Fisher Scientific Co. Fair Lawn, N.J.). The grid was examined by an electron microscope operated at 75 kV (Hitachi 117000, Tokyo, Japan). Digital images were taken by a CCD camera (Gatan Inc. Pleasanton, Calif.).

### Flow Cytometry

Phage were incubated with E. coli cells for 4 min at 37° C. 1 μl streptavidin coated Quantum dots (1 μM) were added and incubation continued for 5 min at room temperature. After centrifugation at 1500 rpm for 5 min, 1 μl of the sample was resuspended to 6×10⁴ cells/ml. Samples were analyzed by flow cytometry using BD FACS DNA LSR II (Becton Dickinson) monitoring the ratio of 407/600 nm excitation/emission fluorescence from phage-QDs bound cells. Events shown in histograms were gated on fluorescence. All were detected in log scale, and events were triggered on SSC. A total of 30,000 events were collected for each analysis.

### Fluorescence Microscopy

Samples were prepared as described for flow cytometry, except that an additional centrifugation was performed and 2 μl of the sample were placed on a microscope slide, covered with a cover slip and visualized on an Olympus Vanox-T microscope using an Oriel 500 W Hg arc lamp running at 200 W, a fluorescence filter set (a bandpass exciter (447±15 nm), a dichroic mirror (505 nm cutoff), and a longpass emission filter (560 nm cutoff)), and a 1.25 numerical aperture oil immersion objective (DPlan 100×, Olympus). Images were captured by an intensified cooled CCD camera (I-PentaMAX, Roper Scientific, Inc.).

Detection of E. coli in a Mix of Bacteria

2×10⁶ cells of each of the following strains Pseudomonas aeruginosa, Vibrio cholera, Salmonella enterica serovar Typhimurium, Yersinia pseudotuberculosis, Bacillus subtilis were mixed with different numbers of Escherichia coli BL-21 cells, 10-10⁷, as estimated by OD 600 and confirmed by viable count. After about 10-15 min at 37° C., lysates were cleaned by centrifugation and assayed using the fluorescence microscope.

### Example 2

### Engineered Phage Containing birA Gene and Biotinylation Domain

The birA gene was engineered into phage along with a biotinylation domain to allow more of the displayed peptides to be biotinylated, thereby increasing the detection sensitivity. The BPL of E. coli, birA gene, was clone as a transcriptional fusion with the phage Capsid-bio under the phage promoter.

This engineered phage (capsid-bio-birA) had about a 100 fold higher level of biotinylation than the Capsid-bio engineered phage as judged by western blot analysis with streptavidin-HRP. This new engineered phage overcomes potential limitation of the endogenous BirA protein such that most of the displayed biotinylation domain becomes biotinylated.

### Example 3

### Phage-QD Complexes and Analysis of Quantized Levels of QDs in Complexes Binding to Bacteria

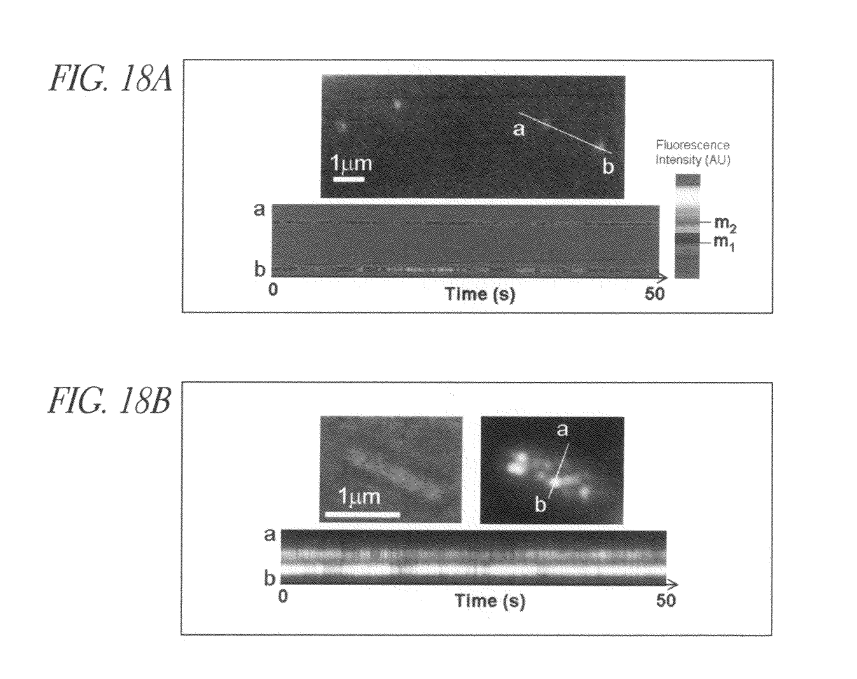

A fluorescence image of phage-QD complexes' spread on a glass coverslip is shown in FIG. 18A (top). Each bright spot in the image exhibits fluorescence signal from one or two of QDs attached onto different phage. The image was time-averaged from 500 movie frames taken at the rate of 100 ms per frame. In FIG. 18A (bottom), a time-transient intensity along the line of (a-b) shows that the fluorescent spot near “a” shows a single level quantized blinking indicative of one QD, while the other fluorescent spot near “b” shows two-levels of quantized blinking from two QDs. m1 and m2 in the intensity scale bar correspond to two local maxima of the (occurrence vs. intensity) histogram calculated from the intensity fluctuation of the 2 QD spot.

Time-averaged bright field and fluorescence imaging of bacteria cells was done after an attempt to bind maximum number of phage-QD complexes by adding excess number of phage-QD complexes (FIG. 18B). Fluorescence time-transient intensity is measured along the line (a-b) as shown in FIG. 18B, top right and bottom panels. The number of quantized levels in the time transient plots measures the number of QDs in each single phage-QD complex shown as a diffraction-limited bright spot.

### Example 4

### Category A

Detection of Yersinia pestis with phiA1122

Recombinant, non-biotinylated phiA1122-bio and phiA1122-myc phages are engineered using standard molecular biology protocols. PhiA1122 grows on almost all isolates of Yersinia pestis. Phage are incubated with Y. pestis cells for 4 min at 37° C. Streptavidin coated Quantum dots (1 μM) are added and incubation continued for 5 min at room temperature. After centrifugation at 1500 rpm for 5 min, 1 μl of the sample is resuspended to 6×10⁴ cells/ml. Samples are analyzed by flow cytometry using BD FACS DIVA LSR II (Becton Dickinson) monitoring the ratio of 407/600 nm excitation/emission fluorescence from phage-QDs bound cells. Events shown in histograms are gated on fluorescence. All are detected in log scale, and events are triggered on SSC. A total of 30,000 events are collected for each analysis.

Samples may alternatively be detected by fluorescence microscopy as described in Example 1 and Yersinia pestis is detected in a mix of bacteria as described in Example 1.

### Example 5

### Category B

Detection of Escherichia coli (O157:H7) with PP01 Bacteriophage

Recombinant, non-biotinylated PP01-bio and PP01-myc phages are engineered using standard molecular biology protocols. The virulent phage PP01 infects E. coli O157:H7 strains with high specificity (Morita M. et al. 2002 FEMS Microbiol Lett 216:243-248). Phage are incubated with E. coli cells for 4 min at 37° C. Streptavidin coated Quantum dots (1 μM) are added and incubation continued for 5 min at room temperature. After centrifugation at 1500 rpm for 5 min, 1 μl of the sample is resuspended to 6×10⁴ cells/ml. Samples are analyzed by flow cytometry using BD FACS DIVA LSR II (Becton Dickinson) monitoring the ratio of 407/600 nm excitation/emission fluorescence from phage-QDs bound cells. Events shown in histograms are gated on fluorescence. All are detected in log scale, and events are triggered on SSC. A total of 30,000 events are collected for each analysis.

Samples may alternatively be detected by fluorescence microscopy as described in Example 1 and E. coli is detected in a mix of bacteria as described in Example 1.

### Example 6

### Category C

Detection of Mycobacterium tuberculosis by D29

Recombinant, non-biotinylated D29-bio and D29-myc phages are engineered using standard molecular biology protocols. D29 is a lytic, double-stranded DNA phage with a wide mycobacterial host range. Phage are incubated with E. coli cells for 4 min at 37° C. Streptavidin coated Quantum dots (1 μM) are added and incubation continued for 5 min at room temperature. After centrifugation at 1500 rpm for 5 min, 1 μl of the sample is resuspended to 6×10⁴ cells/ml. Samples are analyzed by flow cytometry using BD FACS DIVA LSR II (Becton Dickinson) monitoring the ratio of 407/600 nm excitation/emission fluorescence from phage-QDs bound cells. Events shown in histograms are gated on fluorescence. All are detected in log scale, and events are triggered on SSC. A total of 30,000 events are collected for each analysis.

Samples may alternatively be detected by fluorescence microscopy as described in Example 1 and M. tuberculosis is detected in a mix of bacteria as described in Example 1.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

What is claimed is:

1. A non-biotinylated bacteriophage that comprises a nucleic acid sequence encoding a biotinylation domain.

2. The bacteriophage of claim 1 wherein said bacteriophage is specific for a Category A bacteria.

3. The bacteriophage of claim 2 wherein said bacteriophage is specific for Yersinia pestis.

4. The bacteriophage of claim 1 wherein said bacteriophage is specific for a Category B bacteria.

5. The bacteriophage of claim 4 wherein said bacteriophage is specific for strain
O157:H7.

6. The bacteriophage of claim 1 wherein said bacteriophage is specific for a Category C bacteria.

7. The bacteriophage of claim 6 wherein said bacteriophage is specific for multi-drug resistant TB.

8. The bacteriophage of claim 1 wherein said biotinylation domain comprises SEQ ID NO: 1.

9. The bacteriophage of claim 8 wherein said biotinylation domain comprises SEQ ID NO: 4.

10. A complex that comprises:
a) a biotinylated bacteriophage, and
b) a biotin-specific ligand conjugated bioconjugate.

11. The complex of claim 10 wherein the bioconjugate comprises a fluorescent semiconductor nano crystal.

12. The complex of claim 10 wherein the biotin-specific ligand is streptavidin.

13. A method of detecting a bacterial cell in a sample comprising:
contacting the sample with a non-biotinylated bacteriophage that comprises a nucleic acid sequence encoding a biotinylation domain, wherein the bacteriophage is specific to the bacterial cell.

14. The method of claim 13 further comprising:
incubating the sample under conditions effective to form biotinylated bacteriophage, and
detecting the presence of biotinylated bacteriophage by addition of a biotin-specific ligand conjugated bioconjugate, wherein the presence of biotinylated bacteriophage in the sample indicates the presence of target bacterial cells in the sample.

15. The method of claim 13 wherein said biotinylation domain comprises SEQ ID NO: 1.

16. The method of claim 15 wherein said biotinylation domain comprises SEQ ID NO: 4.

17. The method of claim 14 wherein the bioconjugate comprises a fluorescent semiconductor nanocrystal.

18. The method of claim 14 wherein the biotin-specific ligand is streptavidin.

19. A bacteriophage engineered to have the major head, capsid protein assembly of the phage express a first attachment site.

20. A complex that comprises:
a) a bacteriophage engineered to have the major head, capsid protein assembly of the phage express a first attachment site, and
b) a bioconjugate functionalized with a second attachment site capable of recognizing/binding the first attachment, site expressed on the engineered phage.


SEQUENCE LISTING (9 sequences)

SEQ ID NO: 1 - protein, 13 aa, Artificial Sequence
  Synthetic consensus sequence
LXXIXXXXKXXXX

SEQ ID NO: 2 - protein, 23 aa, Artificial Sequence
  Synthetic peptide
MAGGLNDIFEAQKIEWHEDTGGS

SEQ ID NO: 3 - protein, 14 aa, Artificial Sequence
  Synthetic peptide
GLNDIFEAQKIEWH

SEQ ID NO: 4 - protein, 15 aa, Artificial Sequence
  Synthetic Peptide
GLNDIFEAQKIEWHE

SEQ ID NO: 5 - protein, 10 aa, Artificial Sequence
  Synthetic peptide
EQKLISEEDL

SEQ ID NO: 6 - DNA, 70 bp, Artificial Sequence
  Synthetic oligonucleotide
agcttttagtgccattcgattttctgagcttcgaagatgtcgttcaggcctgaaccacgcggccgcaacg

SEQ ID NO: 7 - DNA, 70 bp, Artificial Sequence
  Synthetic oligonucleotide
aattcgttgcggccgcgtggttcaggcctgaacgacatcttcgaagctcagaaaatcgaatggcactaaa

SEQ ID NO: 8 - DNA, 58 bp, Artificial Sequence
  Synthetic oligonucleotide
aattctggtggcagcggatctgagcagaagctgatcagcgaggaagatcttaattaaa

SEQ ID NO: 9 - DNA, 57 bp, Artificial Sequence
  Synthetic oligonucleotide
agctttaattaagatcttcctcgctgatcagcttctgctcagatccgctgccaccag